(19) United States
(12) Patent Application Publication
(10) Pub. No.: US 2024/0197730 A1
(43) Pub. Date: Jun. 20, 2024

(54) CANCER TREATMENT USING PARP INHIBITORS AND PLK1 INHIBITORS

(71) Applicant: Cardiff Oncology, Inc., San Diego, CA (US)

(72) Inventors: Maya RIDINGER, San Diego, CA (US); Mark ERLANDER, San Diego, CA (US)

(21) Appl. No.: 18/554,158

(22) PCT Filed: Apr. 8, 2022
(86) PCT No.: PCT/US2022/024036

§ 371 Date: Oct. 5, 2023

Related U.S. Application Data
(60) Provisional application No. 63/322,557, filed on Mar. 22, 2022.
(60) Provisional application No. 63/182,674, filed on Apr. 30, 2021.
(60) Provisional application No. 63/173,278, filed on Apr. 9, 2021.

(51) Int. Cl. A61K 31/502; A61K 31/517; A61P 35/00

(52) U.S. Cl. CPC A61K 31/502; A61K 31/517; A61P 35/00

(57) ABSTRACT

Provided include methods, compositions and kits for treating cancer in a subject. The method can comprise administrating a PARP inhibitor (for example, olaparib) and a PLK1 inhibitor (for example, onvansertib) to the subject in a manner sufficient to inhibit progression of the cancer.

### RELATED APPLICATIONS

The present application is a U.S. national phase application under 35 U.S.C. § 371 of International Application No. PCT/US2022/024036, filed on Apr. 8, 2022 and published as WO 2022/217060 A1 on Oct. 13, 2022, which claims priority under 35 U.S.C. § 119(e) to U.S. Provisional Application No. 63/173,278, filed Apr. 9, 2021; U.S. Provisional Application No. 63/182,674, filed Apr. 30, 2021; and U.S. Provisional Application No. 63/322,557, filed Mar. 22, 2022, the content of these related applications is herein expressly incorporated by reference in its entirety.

### BACKGROUND

### Field

The present application generally relates to treatment for cancer. More specifically, combination therapies for treating cancer using PARP inhibitors in combination with polo-like kinase 1 (PLK1) inhibitors are provided.

### Description of the Related Art

The Polo-like kinase 1 (PLK1) is a well characterized member of the 5 members of the family of serine/threonine protein kinases and strongly promotes the progression of cells through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is a master regulator of mitosis and aberrantly overexpressed in a variety of human cancers including AML and is correlated with cellular proliferation and poor prognosis.

PARP inhibitors are inhibitors of the enzyme poly(ADP-ribose) polymerase (PARP). Poly(ADP-ribose) polymerase 1 (PARP1) is a key molecule in the repair of DNA single-strand breaks (SSBs). Knockout of SSB repair by inhibition of PARP1 function induces DNA double-strand breaks (DSBs) that can trigger synthetic lethality in cancer cells with defective homology-directed DSB repair. Some PARP inhibitors have been approved for treating certain cancer types, but patients can be resistant or develop resistance to PARP inhibitor treatment.

There is a need to find effective treatment for cancer patients, including the patients resistant to PARP inhibitor treatment.

### SUMMARY

Provided include methods, compositions and kits for treating cancer. Some embodiments provide a method of treating cancer comprises administrating a Poly-(ADP-ribose) polymerase (PARP) inhibitor and a Polo-like kinase 1 (PLK1) inhibitor to a subject with cancer, thereby inhibiting cancer progression. In some embodiments, the subject has ovarian cancer, breast cancer, prostate cancer, colorectal cancer, pancreatic cancer, or a combination thereof. The cancer can be, for example, a homologous recombination-deficient cancer. The cancer can be, for example, a BRCA1-mutant cancer, a BRCA2-mutant cancer, or both. In some embodiments, the subject is human. In some embodiments, the subject is resistant to, or has been developed resistance to, PARP inhibitor treatment alone. The patient's resistance to PARP inhibitor treatment can be partial lack of response, or a complete lack of response to the PARP inhibitor treatment alone. The PLK1 inhibitor and the PARP inhibitor can be co-administered simultaneously, or administered sequentially. In some embodiments, the PLK1 inhibitor is administered prior to the administration of the PARP inhibitor, and optionally wherein the PLK1 inhibitor is administered prior to the administration of the PARP inhibitor every day on which the subject is administered with the PLK1 inhibitor and the PARP inhibitor. In some embodiments, the PLK1 inhibitor is administered about 30 minutes to about 5 hours prior to the administration of the PARP inhibitor on a given day. In some embodiments, the administration of the PLK1 inhibitor is oral administration, the administration of the PARP inhibitor is oral administration, or both.

In some embodiments, the inhibition of cancer progression is greater than the combined inhibition of progression caused by the PARP inhibitor alone plus the PLK1 inhibitor alone. In some embodiments, the subject achieves a complete response. In some embodiments, the subject has received a prior PARP inhibitor treatment. In some embodiments, the subject did not respond to treatment with the PARP inhibitor alone. In some embodiments, subject is known to be resistant to a PARP inhibitor therapy. In some embodiments, the PARP inhibitor and the PLK1 inhibitor are each administered to the subject in a cycle of at least twice or at least five times within a week. In some embodiments, the PARP inhibitor, the PLK1 inhibitor, or both are administered in a cycle of at least 7 days. A cycle of treatment (e.g., each cycle of the treatment) can be at least about 21 days, for example from about 21 days to about 28 days. In some embodiments, the PLK1 inhibitor is administered on at least four days in the cycle. In some embodiments, the PLK1 inhibitor is not administered on at least one day in the cycle. In some embodiments, the PARP inhibitor is administered daily.

In some embodiments, the subject undergoes at least two cycles of the administration of the PARP inhibitor and the PLK1 inhibitor. In some embodiments, the PARP inhibitor is selective and/or specific for PARP inhibition. Non-limiting examples of the PARP inhibitor include iniparib (BSI 201), talazoparib (BMN-673), olaparib (AZD-2281), AZD5305, NMS-293, rucaparib (AG014699, PF-01367338), ABT-888, Veliparib (ABT-888), niraparib, CEP 9722, MK 4827, BGB-290 (pamiparib), BSI-201, CEP-8983, E7016, 3-aminobenzamide, or a combination thereof; optionally the PARP inhibitor is olaparib. In some embodiments, the PLK1 inhibitor is selective and/or specific for PLK1. Non-limiting examples of the PLK1 inhibitor include a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280; and optionally the PLK1 inhibitor is onvansertib.

Onvansertib can be administered to the subject at 12 mg/m²-90 mg/m². In some embodiments, a maximum concentration (Cₘₐₓ) of onvansertib in a blood of the subject is from about 100 nmol/L to about 1500 nmol/L. In some embodiments, an area under curve (AUC) of a plot of a concentration of onvanserib in a blood of the subject over time is from about 1000 nmol/L.hour to about 400000 nmol/L.hour. In some embodiments, a time (Tₘₐₓ) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours.

In some embodiments, the PARP inhibitor is olaparib or NMS-293, and the PLK1 inhibitor is onvansertib. In some embodiments, the subject has received at least one prior cancer treatment. In some embodiments, the prior treatment does not comprise the use of a PARP inhibitor, a PLK inhibitor, or both. In some embodiments, the subject was in remission for cancer, optionally wherein the subject in remission for cancer was in complete remission (CR) or in partial remission (PR). The method disclosed herein can further comprise one or more of (1) determining cancer status of the subject, (2) determining responsiveness of the subject to a PLK1 inhibitor treatment, and (3) administering one or more cancer therapeutics or therapies for the cancer. In some embodiments, the subject is human.

Disclosed herein include a method of sensitizing cancer cells to a PARP inhibitor. In some embodiments, the method comprises: contacting cancer cells with a composition comprising a Polo-like kinase 1 (PLK1) inhibitor, thereby sensitizing the cancer cells to the PARP inhibitor.

In some embodiments, the PLK1 inhibitor is onvansertib, the PARP inhibitor is olaparib, or both. In some embodiments, contacting cancer cells with the composition occurs in vitro, ex vivo, and/or in vivo. In some embodiments, contacting cancer cells with the composition is in a subject, and optionally wherein the subject did not respond to, or is known to be resistant to, the PARP inhibitor. In some embodiments, the subject had prior treatment with the PARP inhibitor. In some embodiments, the subject is a mammal, for example human.

In some embodiments, the method comprises determining sensitization of the cancer cells to the PARP inhibitor after being contacted with the composition. In some embodiments, the method comprises contacting the cancer cells with the PARP inhibitor, for example contacting the cancer cells with the PARP inhibitor occurs in the subject. In some embodiments, the method comprises determining the response of the subject to the PARP inhibitor. In some embodiments, contacting the cancer cells with the PARP inhibitor is concurrent with the contacting the cancer cells with the composition, or after the contacting the cancer cells with the composition.

Also disclosed herein include a kit, comprising a Polo-like kinase 1 (PLK1) inhibitor; and a manual providing instructions for co-administrating the PLK1 inhibitor with a Poly-(ADP-ribose) polymerase (PARP) inhibitor to a subject for treating cancer. In some embodiments, the subject has ovarian cancer, breast cancer, prostate cancer, colorectal cancer, pancreatic cancer, or a combination thereof. The cancer can be, for example, a homologous recombination (HR)-deficient cancer. In some embodiments, the cancer is a BRCA1-mutant cancer, a BRCA2-mutant cancer, or both.

The instructions can, for example, comprise instructions for co-administrating the PLK1 inhibitor and the PARP inhibitor simultaneously, or instructions for co-administrating the PLK1 inhibitor and the PARP inhibitor sequentially. In some embodiments, the instructions comprise (1) instructions for administering of the PLK1 inhibitor orally, (2) instructions for administrating the PARP inhibitor orally, or both.

In some embodiments, the instructions comprise instructions the subject has received a prior PARP inhibitor treatment. In some embodiments, the instructions comprise instructions the subject did not respond to treatment with the PARP inhibitor alone. In some embodiments, the instructions comprise instructions the subject is known to be resistant to a PARP inhibitor therapy. In some embodiments, the instructions comprise instructions for administering each of the PARP inhibitor and the PLK1 inhibitor to the subject in a cycle of at least twice or at least five times within a week.

In some embodiments, the instructions comprise instructions for administering the PARP inhibitor, the PLK1 inhibitor, or both are in a cycle of at least 7 days. In some embodiments, each cycle of treatment is at least about 21 days, for example each cycle of treatment is from about 21 days to about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the PARP inhibitor daily. In some embodiments, the instructions comprise instructions for administrating the PARP inhibitor and the PLK1 inhibitor for at least two cycles.

In some embodiments, the PARP inhibitor is selective and/or specific for PARP1 and/or PARP2 inhibition. Non-limiting examples of the PARP inhibitor include iniparib (BSI 201), talazoparib (BMN-673), AZD5305, olaparib (AZD-2281), rucaparib (AG014699, PF-01367338), ABT-888, veliparib (ABT-888), niraparib, CEP 9722, MK 4827, BGB-290 (pamiparib), BSI-201, CEP-8983, E7016, 3-aminobenzamide, NMS-P293, or a combination thereof. In some embodiments, the PARP inhibitor is olaparib or NMS-293.

In some embodiments, the PLK1 inhibitor is selective and/or specific for PLK1. Non-limiting examples of the PLK1 inhibitor include a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280; and optionally wherein the PLK1 inhibitor is onvansertib.

In some embodiments, the instructions comprise instructions for administering onvansertib at 12 mg/m²-90 mg/m². In some embodiments, the PARP inhibitor is olaparib or NMS-293, and the PLK1 inhibitor is onvansertib. In some embodiments, the instructions comprise instructions the subject has received at least one prior treatment for the cancer, and optionally wherein the prior treatment does not comprise the use of a PARP inhibitor, a PLK inhibitor, or both. In some embodiments, the instructions comprise instructions the subject was in remission for cancer, and optionally wherein the subject in remission for cancer was in complete remission (CR) or in partial remission (PR). In some embodiments, the kit further comprises the PARP inhibitor.

### BRIEF DESCRIPTION OF THE DRAWINGS

FIG. 1 is a non-limiting schematic illustration showing combining onvansertib and PARP inhibitors in cancer treatment.

FIG. 2 shows a non-limiting dosing schedule used in Example 1.

FIG. 3A is a Kaplan Meier curve showing the probability of survival in a BRCA1-mutant high grade serious ovarian cancer (HGSOC) patient-derived xenograft (PDX) model #HOC22 treated with control, olaparib, onvansertib, or onvansertib and olaparib combination. FIG. 3B is a Kaplan Meier curve showing the probability of survival in an ovarian BRCA1-mutant HGSOC PDX model #HOC266 treated with control, olaparib, onvansertib, or onvansertib and olaparib combination.

FIG. 4A-B are plots showing changes in tumor volume in a BRCA1-wildtype (WT) HGSOC PDX model #HOC124 treated with control, olaparib, onvansertib, or onvansertib and olaparib combination.

FIG. 5 is a table showing molecular and pharmacological characteristics of three PDX models (MNHOC22, MNHOC266 and MNHOC316DDP) used in an exemplary embodiment.

FIG. 6A is a plot showing changes in mice body weight in a MNHOC22 model treated with control, olaparib, onvansertib, or onvansertib and olaparib combination. FIG. 6B is a Kaplan Meier curve showing the probability of survival in the MNHOC22 model treated with control, olaparib, onvansertib, or onvansertib and olaparib combination. FIG. 6C is a plot showing changes in mice body weight in a MNHOC66 model treated with control, olaparib, onvansertib, or onvansertib and olaparib combination. FIG. 6D is a Kaplan Meier curve showing the probability of survival in the MNHOC66 model treated with control, olaparib, onvansertib, or onvansertib and olaparib combination. FIG. 6E is a plot showing changes in mice body weight in a MNHOC316DDP model treated with control, olaparib, onvansertib, or onvansertib and olaparib combination. FIG. 6F is a Kaplan Meier curve showing the probability of survival in the MNHOC316DDP model treated with control, olaparib, onvansertib, or onvansertib and olaparib combination.

FIG. 7 is a table showing median survival times and increase in life span (ILS) in three PDXs (MNHOC22, MNHOC266 and MNHOC316DDP), treated with control, olaparib, onvansertib, or onvansertib and olaparib combination.

FIG. 8 provides plots showing evaluation of Ki67 positivity (panel A), mitosis (panel B), apoptosis (panel C) and RAD51-foci formation (panel D) in PDXs MNHOC22 and MNHOC266 treated with control, olaparib, onvansertib, or onvansertib and olaparib combination.

FIG. 9 shows levels of pSer10-H3 and pSer139-γH2AX in tumors of PDXs MNHOC22 and MNHOC266 treated with control, olaparib, onvansertib, or onvansertib and olaparib combination.

FIG. 10 is a plot showing suppression of tumor growth in a prostate BRCA2-mutant 22RV1 xenograft model treated with control, olaparib, B12536, or B12536 and olaparib combination.

FIG. 11 depicts two circle graphs showing the cancer types of the cell lines tested in a two-dimensional cell culture (top graph) and a three-dimensional cell culture (bottom graph).

FIG. 12A-B depict onvansertib and olaparib synergy scores in cells cultured in a two-dimensional cell culture (FIG. 12A) and a three-dimensional cell culture (FIG. 12B).

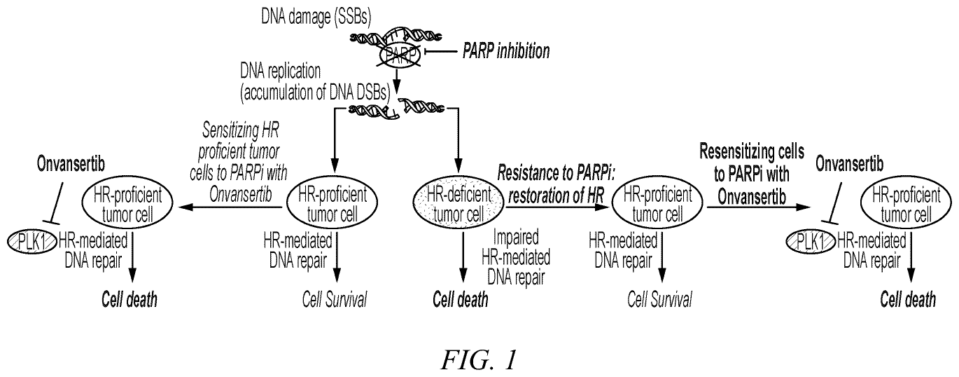

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., PLK1 inhibitor (e.g., onvansertib) or PARP inhibitor (e.g., olaparib)).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response” and “partial remission” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

### Cancer

Methods, compositions and kits disclosed herein can be used for treating cancer. In some embodiments, a method for treating cancer comprises administrating a PARP inhibitor (e.g., olaparib or NMS-293), or a pharmaceutically acceptable salt, solvate, stereoisomer or prodrug thereof, and a Polo-like kinase 1 (PLK1) inhibitor (e.g., onvansertib), or a pharmaceutically acceptable salt, solvate, stereoisomer, or prodrug thereof, to a subject (e.g., a patient) in need thereof.

The methods, compositions and kits disclosed herein can be used to various types of cancer, including but are not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (e.g., prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and Multiple myeloma (“MM”).

The cancer can be, for example, ovarian cancer, breast cancer, prostate cancer, colorectal cancer, pancreatic cancer, or a combination thereof. The cancer can be pancreatic ductal carcinoma, pancreatic adenocarcinoma, ovary serous adenocarcinoma, breast ductal carcinoma, a high-grade serous ovarian adenocarcinoma, or a combination thereof. In some embodiments, the cancer is ovarian cancer. In some embodiments, the cancer is breast cancer. In some embodiments, the cancer is prostate cancer. The cancer can be a BRCA1 mutant cancer, a BRCA2 mutant cancer, or both. In some embodiments, the cancer is a BRCA2-mutant prostate cancer. In some embodiments, the cancer is a BRCA1-mutant ovarian cancer. The cancer can be a BRCA wild type cancer with wildtype BRCA1 and/or BRCA2 sequence, such as BRCA1 wild type ovarian cancer and/or BRCA2 wild type ovarian cancer. In some embodiments, the cancer is a BRCA1-wild type prostate cancer.

In some embodiments, the cancer is sensitive to a PARP inhibitor treatment. In some embodiments, the cancer is characterized by deficiencies in DNA repair. The cancer can be a homologous recombination (HR)-deficient cancer with impaired HR-mediated DNA repair functionality. In some embodiments, the subject having the cancer has one or more pathogenic variants of one or more genes involved in the HR-mediated DNA repair mechanism including but not limited to, BRCA1. BRCA2, 53BP1, ATM, ATR, ATRIP, BARDI, BLM, BRIPI, DMCI, MRE11A, NBN, PALB2, RAD50, RAD51, RAD51B, RAD51C, RAD51D, RIF1, RMI1, RMI2, RPA1, TOP3A, TOPBP1, XRCC2, and XRCC3.

In some embodiments, the cancer is BRCA1- and/or BRCA2-deficient. In some embodiments, HRDetect score can be calculated for a cancer or tumor to detect BRACA1/BRCA2-deficient tumors. HRDetect is a whole-genome sequencing based classifier designed to predict BRCA1 and BRCA2 deficiency based on six mutational signatures. Details about the HRDetect method are described in Davies H et al., Nat Med. 2017; 23:517-25, the content of which is incorporated herein by reference in its entirety. In some embodiments, a HRDetect score equal to or greater than about 0.7 suggest a HR deficiency, while less than about 0.7 indicates HR proficiency. In some embodiments, a cancer has a HRDetect score equal to or greater than 0.7. In some embodiments, a cancer has a HRDetect score less than 0.7.

In some embodiments, the cancer is a PARP inhibitor resistant cancer or has developed resistance to a PARP inhibitor. The cancer can be a PARP inhibitor resistant (e.g., olaparib resistant) breast cancer, a PARP inhibitor resistant (e.g., olaparib resistant) ovarian cancer, a PARP inhibitor resistant (e.g., olaparib resistant) pancreas cancer, or a PARP inhibitor resistant (e.g., olaparib resistant) prostate cancer. In some embodiments, the combinatorial inhibition of PLK1 and PARP can effectively treat PARP inhibitor resistant cancer by significantly reducing the tumor size, increasing cancer survival rate and prolonging cancer survival duration in comparison with a single agent treatment (a PARP inhibitor or a PLK1 inhibitor).

In some embodiments, RAD51 foci assay is conducted to discriminate between PARP inhibitor-sensitive and PARP inhibitor-resistant cancer. RAD51 refers to a homologous recombination DNA repair protein forming nuclear foci after DNA damage and can be used as an indicator of homologous recombination DNA repair functionality. RAD51 foci can be quantified using an immunofluorescence-based method in formalin-fixed paraffin-embedded tumor samples treated with vehicle or a PARP inhibitor. Low RAD51 score can be associated with PARP inhibitor (e.g., olaparib or NMS-293) sensitivity, while high RAD51 score can be associated with PARP inhibitor (e.g., olaparib or NMS-293) resistance. Details about the RAD51 foci assay can be found, for example, in Guffanti F et al., Br J Cancer, 2022 Jan.; 126(1):120-128, the content of which is incorporated by reference herein. In some embodiments, the cancer has a RAD51 foci level equal to or greater than 30%, 35%, 40%, 45%, 50%, 55%, 60%, 65%, 70%, 75%, 80%, 85%, 90%, 95%, or 98%. In some embodiments, the cancer has a RAD51 foci level less than 30%, 25%, 20%, 155, 10%, 5%, 2%, or 1%.

### PARP Inhibitors and PLK Inhibitors

Methods, compositions and kits disclosed herein can be used for treating cancer, for example ovarian cancer, breast cancer, prostate cancer, colorectal cancer, pancreatic cancer, or a combination thereof. In some embodiments, a method for treating cancer comprises administrating a PARP inhibitor (e.g., olaparib or NMS-293), or a pharmaceutically acceptable salt, solvate, stereoisomer, or prodrug thereof, and a PLK1 inhibitor (e.g., onvansertib), or a pharmaceutically acceptable salt, solvate, stereoisomer, or prodrug thereof, to a subject (e.g., a patient) in need thereof. The method can comprise administering a pharmaceutically effective amount of the PARP inhibitor and a pharmaceutically effective amount of the PLK1 inhibitor.

### PARP Inhibitors

Poly-(ADP-ribose) polymerase (PARP) plays a key role in the DNA damage response and either directly or indirectly affects numerous DDR pathways, including BER, HR, NER, NHEJ and MMR. PARP inhibitors are inhibitors of PARP, which are developed for multiple indications, including treatment of cancer. PARP is essential for repair of single strand DNA breaks (SSBs). Failure to repair SSBs through PARP inhibition results in double strand DNA breaks (DSBs). In cells with functional homologous recombination (HR) pathway, the DSB are repaired. In cells with a dysfunctional HR pathway, such as BRCA1 and/or BRCA2 mutant cells, the lesions cannot be adequately repaired resulting in cell death.

Several forms of cancer are more dependent on PARP than regular cells, making PARP an attractive target for cancer therapy. In addition to their use in cancer therapy, PARP inhibitors are considered a potential treatment for acute life-threatening diseases, such as stroke and myocardial infarction, as well as for long-term neurodegenerative diseases. DNA is damaged thousands of times during each cell cycle, and that damage must be repaired. BRCA1, BRCA2 and PALB2 are proteins that are important for the repair of double-strand DNA breaks by the error-free homologous recombination repair, or HRR, pathway. When the gene for either protein is mutated, the change can lead to errors in DNA repair that can eventually cause breast cancer. When subjected to enough damage at one time, the altered gene can cause the death of the cells. PARP1 is a protein that is important for repairing single-strand breaks (“nicks” in the DNA). If such nicks persist unrepaired until DNA is replicated (which must precede cell division), then the replication itself can cause double strand breaks to form. Drugs that inhibit PARP1 cause multiple double strand breaks to form in this way, and in tumors with BRCA1, BRCA2 or PALB2 mutations these double strand breaks cannot be efficiently repaired, leading to the death of the cells. Normal cells that don't replicate their DNA as often as cancer cells, and that lacks any mutated BRCA1 or BRCA2 still have homologous repair operating, which allows them to survive the inhibition of PARP. Some cancer cells that lack the tumor suppressor PTEN may be sensitive to PARP inhibitors because of down-regulation of Rad51, a critical homologous recombination component, although other data suggest PTEN may not regulate Rad51. In some embodiments, PARP inhibitors are PARP inhibitors effective against one or more PTEN-defective tumors (e.g. some aggressive prostate cancers). Cancer cells that are low in oxygen (e.g. in fast growing tumors) are sensitive to PARP inhibitors. PARP inhibitors were originally thought to work primarily by blocking PARP enzyme activity, thus preventing the repair of DNA damage and ultimately causing cell death. PARP inhibitors have an additional mode of action: localizing PARP proteins at sites of DNA damage, which has relevance to their anti-tumor activity. The trapped PARP protein-DNA complexes are highly toxic to cells because they block DNA replication. The PARP family of proteins in humans includes PARP1 and PARP2, which are DNA binding and repair proteins. When activated by DNA damage, these proteins recruit other proteins that do the actual work of repairing DNA. Under normal conditions, PARP1 and PARP2 are released from DNA once the repair process is underway. But when they are bound to PARP inhibitors, PARP1 and PARP2 become trapped on DNA. It was shown that trapped PARP-DNA complexes are more toxic to cells than the unrepaired single-strand DNA breaks that accumulate in the absence of PARP activity, indicating that PARP inhibitors act as PARP poisons. As described herein, there are two classes of PARP inhibitors: (1) catalytic inhibitors that act mainly to inhibit PARP enzyme activity and do not trap PARP proteins on DNA, and (2) dual inhibitors that both block PARP enzyme activity and act as PARP poison. Non-limiting examples of PARP inhibitors include: Iniparib (BSI 201) (for example, for breast cancer and squamous cell lung cancer); Olaparib (AZD-2281) (for example, for breast, ovarian and colorectal cancer); Rucaparib (AG014699, PF-01367338, for example, for metastatic breast and ovarian cancer); Veliparib (ABT-888) (for example, for metastatic melanoma and breast cancer); CEP 9722 (for example, for non-small-cell lung cancer (NSCLC)); MK 4827 which inhibits both PARP1 and PARP2; BMN-673 (for example, for advanced hematological malignancies and for advanced or recurrent solid tumors); and 3-aminobenzamide. The PARP inhibitor can be a PARP1 inhibitor or a PARP2 inhibitor. In some embodiments, the PARP inhibitor can inhibit PARP1 and PARP2. The PARP inhibitor can be a selective inhibitor for PARP1, PARP2, or both.

The methods and compositions for treating cancer in combination with one or more PLK1 inhibitors disclosed herein can include one or more PARP inhibitors (including but not limited to, olaparib, talazoparib (BMN-673), AZD5305, rucaparib, veliparib, niraparib, CEP 9722, MK 4827, BGB-290 (pamiparib), ABT-888, AG014699, BSI-201, CEP-8983, E7016, NMS-P293, and 3-aminobenzamide). PARP inhibitors are known to exhibit synthetic lethality, for example in tumors with mutations in BRCA½. Olaparib has received FDA approval for treatment of ovarian cancer patients with mutations in BRCA1 or BRCA2. In addition to olaparib, other FDA-approved PARP inhibitors for ovarian cancer include nirapirib and rucaparib. Talazoparib was recently approved for treatment of breast cancer with germline BRCA mutations and is in phase III trials for hematological malignancies and solid tumors and has reported efficacy in SCLC, ovarian, breast, and prostate cancers. Veliparib is in phase III trials for advanced ovarian cancer, TNBC and NSCLC. Not all PARP inhibitors are dependent on BRCA mutation status and niraparib has been approved for maintenance therapy of recurrent platinum sensitive ovarian, fallopian tube or primary peritoneal cancer, independent of BRCA status. NMS-P293 was described in, for example, Abstract 4843: NMS-P293, a PARP-1 selective inhibitor with no trapping activity and high CNS penetration, possesses potent in vivo efficacy and represents a novel therapeutic option for brain localized metastases and glioblastoma, Proceedings: AACR Annual Meeting 2018; Apr. 14-18, 2018; Chicago, IL.

Some PARP inhibitors have been approved for BRCA½ mutant ovarian, breast, prostate and pancreatic cancer patients. Although initial response to PARP inhibitors is high, patients will eventually develop resistance. Mechanisms of resistance to PARP inhibitors include restoration of homologous recombination (HR).

The PARP inhibitor can be, for example, Iniparib (BSI 201), Talazoparib (BMN-673), AZD5305, Olaparib (AZD-2281), Rucaparib (AG014699, PF-01367338), ABT-888, Veliparib (ABT-888), niraparib, CEP 9722, MK 4827, BGB-290 (pamiparib), BSI-201, CEP-8983, E7016, 3-aminobenzamide, or a combination thereof. In some embodiments, the PARP inhibitor is olaparib.

PLK1 Inhibitors

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 inhibition induces G2-M-phase arrest with subsequent apoptosis in cancer cells, and has emerged as a promising targeted therapy. Several PLK inhibitors have been studied in clinical trials. In a randomized phase II study of patients with AML who were treatment naïve yet unsuitable for induction therapy, the pan-PLK inhibitor, volasertib (BI6727), administered intravenously in combination with LDAC showed a significant increase in OS when compared with LDAC alone. A subsequent randomized phase III study identified no benefit of the combination and described an increased risk of severe infections. PLK1 facilitates HR during Double Strand DNA Break (DSB) Repair. PLK1 phosphorylates Rad51 and BRCA1, facilitating their recruitment to DSB sites and thereby HR-mediated DNA repair.

Onvansertib (also known as PCM-075, NMS-1286937, NMS-937, “compound of formula (I)” in U.S. Pat. No. 8,927,530, IUPAC name 1-(2-hydroxyethyl)-8-{[5-(4-methylpiperazin-1-yl)-2-(trifluoromethoxy) phenyl]amino}-4,5-dihydro-1H-pyrazolo[4,3-h]quinazoline-3-carboxamide) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib is the first PLK1 specific ATP competitive inhibitor administered by oral route to enter clinical trials with proven antitumor activity in different preclinical models. Onvansertib inhibited cell proliferation at nanomolar concentrations in AML cell lines and tumor growth in xenograft models of AML. Onvansertib also significantly increased cytarabine antitumor activity in disseminated models of AML.

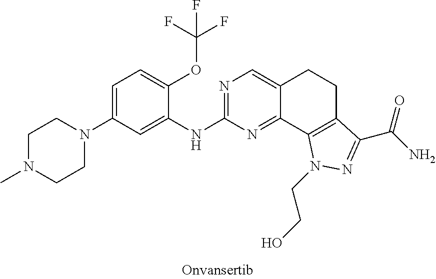 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, both from solid as well as hematologic tumors. Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib (B1I6727, another PLK1 inhibitor), including a higher degree of potency and specificity for the PLK1 isozyme, and oral bioavailability.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in WO 2021/146322, the content of which is incorporated herein by reference in its entirety.

As disclosed herein, a combinations therapy of a PARP inhibitor and a PLK1 inhibitor (including onvansertib) can result in significantly enhanced efficacy against cancer (e.g., breast cancer, ovarian cancer, colorectal cancer, prostate cancer, head and neck cancer, non-small cell lung cancer, intrahepatic cholangiocarcinoma, gastric cancer, urothelial cancer, small cell lung cancer, endometrial cancer, cervical cancer, rhabdomyosarcoma, cholangiocarcinoma, or a combination thereof), causing tumor regression and cancer survival. The resulted tumor regression and cancer survival rate/duration by the combination can be surprisingly synergistic (i.e., more than additive, superior to the cumulated anti-tumor efficacy caused by the PARP inhibitor and the PLK1 inhibitor separately). The PLK1 inhibitor can be onvansertib. Provided herein include methods, compositions and kits for treating cancer in a subject (e.g., a human patient suffering from cancer). The method comprises administrating a PARP inhibitor and a PLK1 inhibitor to the patient in a manner sufficient to inhibit or reduce progression of the cancer. For example, the PARP inhibitor and the PLK1 inhibitor can be administrated to a subject with cancer simultaneously, separately, or sequentially. Surprisingly, the resulted tumor regression and cancer survival rate/duration by the combination is more than additive, i.e., superior to the cumulated anti-tumor efficacy caused by the PARP inhibitor and the PLK1 inhibitor separately. Provided herein include methods, compositions and kits for treating cancer in a subject (for example, a human patient suffering from cancer). The method comprises administrating a PARP inhibitor and a PLK1 inhibitor to the patient in a manner sufficient to inhibit progression of the cancer. For example, the PARP inhibitor and the PLK1 inhibitor can be administrated to a subject with cancer simultaneously, separately, or sequentially.

In some embodiments, the inhibition or reduction of cancer progression is not merely additive, but is enhanced or synergistic (that is, the inhibition is greater than the combined inhibition of progression caused by the PARP inhibitor alone plus the PLK1 inhibitor alone). The enhanced or synergistic efficacy or inhibition of any combination of a PARP inhibitor and a PLK1 inhibitor of the present disclosure can be different in different embodiments. In some embodiments, the enhanced or synergistic efficacy or inhibition of any combination of a PARP inhibitor and a PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the combined inhibition of progression caused by the PARP inhibitor alone plus the PLK1 inhibitor alone.

The molar ratio of the PLK1 inhibitor (e.g., onvansertib) to the PARP inhibitor (e.g., olaparib or NMS-293) can be, for example, about 1:200, 1:100, 1:90, 1:80, 1:70, 1:60, 1:50, 1:40, 1:30, 1:20, 1:10, 1:1, 10:1, 20:1, 30:1, 40:1, 50:1, 100:1, 1000:1, 2000:1, or 5000:1, or a number or a range between any two of these values. In some embodiments, the enhanced or synergistic efficacy or inhibition of cancer progression caused by a combination of the PARP inhibitor and the PLK1 inhibitor (e.g., onvansertib) is, is about, is at least, is at least about, is at most, or is at most about 20%, 30%, 40%, 50%, 60%, 70%, 80%, 90%, 100%, 110%, 120%, 130%, 140%, 150%, 160%, 170%, 180%, 190%, 200%, 250%, 300%, or a number or a range between any two of these values, higher than the combined inhibition of progression caused by the PARP inhibitor alone plus the PLK1 inhibitor (e.g., onvansertib) alone. For example, a combination of the PARP inhibitor and the PLK1 inhibitor can cause a 50%, 60%, 70%, 80%, 90%, or more, inhibition of cancer progression (cancer cell viability of 50%, 40%, 30%, 20%, 10%, or less), whereas under the same conditions the combined inhibition of the PARP inhibitor alone plus the PLK1 inhibitor alone can be 10%, 20%, 25%, 30%, or less) inhibition of cancer progression (cancer cell viability of 90%, 80%, 75%, 70%, or more). Thus, the enhanced or synergistic efficacy or inhibition of cancer progression caused by the combination of the PARP inhibitor and the PLK1 inhibitor for example, 50%, 60%, 70%, 80%, 90%, 100%, or more higher than the combined inhibition of progression caused by the PARP inhibitor alone plus the PLK1 inhibitor alone. In some embodiments, the PARP inhibitor is olaparib and the PLK1 inhibitor is onvansertib.

The method described herein using the combination of the PARP inhibitor and the PLK1 inhibitor is expected to be effective with various cancer, for example head and neck cancer, non-small cell lung cancer, intrahepatic cholangiocarcinoma, gastric cancer, urothelial cancer, small cell lung cancer, breast cancer, endometrial cancer, cervical cancer, rhabdomyosarcoma, cholangiocarcinoma, liver cancer, ovarian cancer, prostate cancer, colorectal cancer, pancreatic cancer, prostate cancer, or a combination thereof.

As described herein, the patient can achieve complete response or partial response after treatment with the PARP inhibitor and the PLK1 inhibitor. In some embodiments, the patient achieves a complete response. In some embodiments, the patient achieves a partial response. In some embodiments, the patient did not respond to treatment with only PARP inhibitor(s). In some embodiments, the patient did not respond to treatment with the PARP inhibitor alone.

The PARP inhibitor and the PLK1 inhibitor can be administered to the patient in any manner deemed effective to treat the cancer. The PARP inhibitor can be administered together with, or separately from, the PLK1 inhibitor. When administered separately, the PARP inhibitor can be administered before or after the PLK1 inhibitor, or in different administration cycles.

The PLK1 inhibitor and the PARP inhibitor can be co-administered (i.e., simultaneously) or sequentially. In some embodiments, it can be advantageous to administer the PLK1 inhibitor (e.g., onvansertib) to the subject before the PARP inhibitor (e.g., olaparib or NMS-293), e.g., on one or more days, or each day, of the days on which the PLK1 inhibitor and the PAPR inhibitor are administered to the subject, such that the PLK1 inhibitor can sensitize cells (e.g., cancer cells) to the PARP inhibitor (e.g., through impairment of HR) to achieve effective treatment. The time interval between the administration of the PLK1 inhibitor and the administration of the PARP inhibitor can be, for example, 30 minutes, 1 hour, 2 hours, 3 hours, 4 hours, 5 hours, 6 hours, 7 hours, 8 hours, 9 hours, 10 hours, 11 hours, 12 hours, a range between any two of these values, or any value between 30 minutes and 12 hours. In some embodiments, the PLK1 inhibitor (e.g., onvansertib) and the PARP inhibitor (e.g., olaparib) are both administered to the subject on, or on at least about, 50%, 60%, 70%, 75%, 80%, 85%, 90%, 95%, or 100% of the days in a cycle (e.g., in each cycle during the combination treatment), and optionally the PLK1 inhibitor is administered to the subject prior to the PARP inhibitor on each of the days both are administered, for example the PLK1 inhibitor is administered 30 minutes, 1 hour, 2 hours, 3 hours, 4 hours, 5 hours, 6 hours, 7 hours, 8 hours, 9 hours, 10 hours, 11 hours, 12 hours, a range between any two of these values, or any value between 30 minutes and 12 hours, prior to the administration of the PARP inhibitor.

The PARP inhibitor and the PLK1 inhibitor can each be administered in any schedule, e.g., once or multiple times per day or week; once, twice, three times, four times, five times, six times or seven times (daily) per week; for one or multiple weeks; etc. In some embodiments, the PARP inhibitor and the PLK1 inhibitor are each administered to the patient in a cycle of at least twice within a week. In other embodiments, the PARP inhibitor and the PLK1 inhibitor are each administered to the patient in a cycle of at least five times within a week. In further embodiments, the patient undergoes at least two cycles of administration. The patient can undergo one cycle or more than one cycle of administrations, for example, two cycles, three cycles, three cycles, four cycles, five cycles, or more. Two adjacent cycles of administration can be continuous, i.e., no break between the last day of the first cycle and the first day of the second cycle. In some embodiments, two adjacent cycles of administration have a break between them, i.e., an interval between the last day of the first cycle and the first day of the second cycle. The break (i.e., the interval) can be or be at least, one day, two days, three days, five days, seven days, ten days, two weeks, three weeks, four weeks, one month, two months, three months, four months, five months, six months, or a number or a range between any two of these values. In some embodiments, the patient undergoes three or four cycles of administration in which each cycle comprises at least five times within a week (e.g., 5 days per week). Each of the cycle in a multi-cycle administration can have the same dosing schedule, or different. For example, one of the cycle in the multi-cycle administration can be five continuous days of daily administration of the PLK1 inhibitor and PARP inhibitor and two days of break in one week for four weeks, and one or more other cycles in the same multi-cycle administration be 28 continuous days of daily administration of the PLK1 inhibitor and PARP inhibitor in a four-week period.

The PARP inhibitor can be administered to the patient at any appropriate dosage, e.g., a dosage of about, at least or at most 0.1 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, 1500 mg/kg, 2000 mg/kg, or a number between any two of these values. The dosage unit based on the body weight (mg/kg) can be converted to another unit (e.g., mg/m²) using a conversion chart such as the body surface area (BSA) conversion chart as will be understood by a person skilled in the art. In some embodiments, the PARP inhibitor is Olaparib or NMS-293, which is administered at a dosage of about, at least or at most 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, or a number between any two of these values.

The PARP inhibitor can be administrated to the patient once daily, twice daily, or three times daily. In some embodiments, the PARP inhibitor is administered in a cycle of 7-56 days of daily administration. In some embodiments, the PARP inhibitor is administered in a cycle of 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 32 days, 35 days, 42 days, 49 days, or 56 days. In some embodiments, the PARP inhibitor is administered in 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 32 days, 35 days, 42 days, 49 days, or 56 days, in a cycle. In some embodiments, the PARP inhibitor is administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 52, day 53, day 54, day 55, and/or day 56. In some embodiments, the PARP inhibitor is not administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 52, day 53, day 54, day 55, and/or day 56. For example, Olaparib or NMS-293can be administered in a cycle of 5, 6, 7, 8, 9, or 10 days. Olaparib can be administrated daily on each day or on selected days of the administration cycle. In some embodiments, Olaparib is administered in a cycle of 7 days with a daily administration for 5 days (e.g., days 1-5) and no administration for two days (e.g. days 6-7).

Any PARP inhibitor, now known or later discovered, can be used in these methods, including PARP inhibitors that are selective for PARP (e.g., PARP1, PARP2 or both), and PARP inhibitors that also inhibit the activity of other proteins. Nonlimiting examples of PARP inhibitors include Iniparib (BSI 201), Talazoparib (BMN-673), AZD5305, Olaparib (AZD-2281), Rucaparib (AG014699, PF-01367338), ABT-888, Veliparib (ABT-888), niraparib, CEP 9722, MK 4827, BGB-290 (pamiparib), BSI-201, CEP-8983, E7016, 3-aminobenzamide, and combinations thereof. In some embodiments, the PARP inhibitor is 2X 121, ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NMS-P293, NOV-140101, NU 1025, NU 1064, NU 1076, NU1085, olaparib (AZD2281), 0N02231, pamiparib, PD 128763, R 503, R554, rucaparib (RUBRACA) (AG-014699, PF-01367338), SBP 101, SC 101914, simmiparib, talazoparib (BMN-673), AZD5305, veliparib (ABT-888), WW 46, 2-(4-(trifluoromethyl)phenyl)-7,8-dihydro-5H-thiopyrano[4,3-d]pyrimidin-4-ol, or combinations thereof. In some embodiments, the PARP inhibitor is olaparib.

In some embodiments, the PARP inhibitor is NMS-P293. NMS-P293 is a nontrapping, potent and selective PARP-1 inhibitor. NMS-P293 has an excellent preclinical profile including: high in vitro cross-species metabolic stability, lack of cytochrome and drug transporter interaction, low protein binding and excellent pharmacokinetic profile, with low clearance and nearly complete oral bioavailability in both rodents and nonrodents; high brain barrier penetration, superior to competitors, which opens up the opportunity to treat brain tumors and brain metastasis; excellent tumor distribution and prolonged pharmacodynamic effect; high single agent antitumor efficacy, with complete regression of BRCA mutated tumor models and cured mice; synergistic efficacy and tolerability in combination with temozolomide (TMZ) in glioblastoma (GBM) tumor models, including TMZ resistant MGMT hypomethylated GBMs.

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

In some embodiments, the PLK1 inhibitor is onvansertib. In these embodiments, the onvansertib is administered to the patient at any appropriate dosage, e.g., a dosage of less than 12 mg/m², less than or equal to 24 mg/m², or greater than 24 mg/m². In some embodiments, the onvansertib is administered to the patient daily. In additional embodiments, the onvansertib is administered in a cycle of 3-10 days of daily onvansertib administration with 2-16 days with no onvansertib administration. In some embodiments, the onvansertib is administered to the patient in a cycle of at least five times within a week. The patient can undergo two, three, or four cycles of administration. In some embodiments, the patient undergoes four cycles of administration in a cycle of at least five days of daily onvansertib administration with 1-2 days with no onvansertib administration.

In some embodiments, a PLK1 inhibitor alone or in combination with a PARP inhibitor is administrated to a patient who has taken a drug holiday after undergoing one or more cycles of administration. A drug holiday as used herein refers to a period of time when a patient stops taking a PLK1 inhibitor and/or a PARP inhibitor. A drug holiday can be a few days to several months. In some embodiments, the drug holiday can be 1 week, 2 weeks, 3 weeks, 4 weeks, 5 weeks, 6 weeks, 7 weeks, 8 weeks, 9 weeks, 10 weeks, or any value or a range between any two of these values.

As can be appreciated by one of skill in the art, the amount of co-administration of the PARP inhibitor and the PLK1 inhibitor, and the timing of co-administration, can depend on the type (species, gender, age, weight, etc.) and condition of the subject being treated and the severity of the disease or condition being treated. The PARP inhibitor and the PLK1 inhibitor can formulated into a single pharmaceutical composition, or two separate pharmaceutical compositions. The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment, a subject at cancer remission, a subject has received one or more cancer treatment, or a subject suspected of having cancer. The subject can have a stage I cancer, a stage II cancer, a stage III cancer, and/or a stage IV cancer. The cancer can be ovarian cancer, breast cancer, prostate cancer, colorectal cancer, pancreatic cancer, or a combination thereof. The cancer can be a BRCA mutant cancer, for example a BRCA1 mutant cancer, a BRAC2 mutant cancer, or a BRAC1 and BRAC2 mutant cancer. The cancer can be a BRCA wild type cancer (e.g., no BRCA mutation), for example a BRCA1 wild type cancer or a BRCA2 wild type cancer. The methods can further comprise administering an additional therapeutic intervention to the subject. The additional therapeutic intervention can comprise a different therapeutic intervention than administering the PLK1 inhibitor and the PARP inhibitor, an antibody, an adoptive T cell therapy, a chimeric antigen receptor (CAR) T cell therapy, an antibody-drug conjugate, a cytokine therapy, a cancer vaccine, a checkpoint inhibitor, a radiation therapy, surgery, a chemotherapeutic agent, or any combination thereof. The therapeutic intervention can be administered at any time of the treatment, for example at a time when the subject has an early-stage cancer, and wherein the therapeutic intervention is more effective that if the therapeutic intervention were to be administered to the subject at a later time.

Without being bound to any particular theory, it is believed that the PLK1 inhibitor (e.g., onvansertib) can sensitize cells (e.g., cancer cells) to PARP inhibitor treatment (e.g., through impairment of HR) to achieve effective cancer treatment.

### Dosing and Pharmacokinetics

The treatment of the present disclosure can comprise administration of a PLK1 inhibitor (e.g., onvansertib) for a desired duration in one or more cycles of treatment, and administration of a PARP inhibitor.

The treatment can, for example, comprise daily administration of a PARP inhibitor (e.g., olaparib) at, or at about, 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 5 mg, 10 mg, 20 mg, 50 mg, 100 mg, 200 mg, 300 mg, 400 mg, 500 mg, 600 mg, 700 mg, 800 mg, 900 mg, 1000 mg, 1100 mg, 1200 mg, or a number or a range between any two of these values. In some embodiments, the daily dose of the PARP inhibitor (e.g., olaparib) can be adjusted (e.g., increased or decreased with the range) during the treatment of the subject. The daily administration of the PARP inhibitor can be at different amounts on different days or during different weeks. For example, the treatment can comprise daily administration of the PARP inhibitor (e.g., olaparib) at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily administration of the PARP inhibitor (e.g., olaparib) at 300 mg on day 1, 450 mg on day 2, 600 mg on day 3, and 750 mg or 600 mg on day 4 and beyond. In some embodiments, the PARP inhibitor (e.g., olaparib) is administered to the subject orally twice daily (two 150 mg tablets each time), with or without food, for a total daily dose of 600 mg. In some embodiments, the PARP inhibitor (e.g., olaparib) is administered to the subject orally twice daily (one 100 mg tablet and one 150 mg tablets each time), with or without food, for a total daily dose of 500 mg. In some embodiments, the PARP inhibitor (e.g., olaparib) is administered to the subject orally twice daily (two 100 mg tablets each time), with or without food, for a total daily dose of 400 mg.

A maximum concentration (Cₘₐₓ) of the PARP inhibitor (e.g., olaparib or NMS-293) in a blood of the subject (during the treatment and/or after the treatment) when the PARP inhibitor is administered alone or in combination with the PLK1 inhibitor can be from about 0.1 pg/mL (picogram per mL) to about 10 μg/mL (microgram per mL). For example, the Cₘₐₓ of the PARP inhibitor (e.g., olaparib) in a blood of the subject when the PARP inhibitor is administered alone or in combination with the PLK1 inhibitor can be, or be about, 0.1 μg/mL, 0.2 μg/mL, 0.3 μg/mL, 0.4 pg/mL, 0.5 μg/mL, 0.6 μg/mL, 0.7 μg/mL, 0.8 μg/mL, 0.9 μg/mL, 1 μg/mL, 1.1 μg/mL, 1.2 μg/mL, 1.3 μg/mL, 1.4 μg/mL, 1.5 μg/mL, 1.6 μg/mL, 1.7 μg/mL, 1.8 μg/mL, 1.9 μg/mL, 2 μg/mL, 2.1 μg/mL, 2.2 μg/mL, 2.3 μg/mL, 2.4 μg/mL, 2.5 μg/mL, 2.6 μg/mL, 2.7 μg/mL, 2.8 μg/mL, 2.9 μg/mL, 3 μg/mL, 3.1 μg/mL, 3.2 μg/mL, 3.3 μg/mL, 3.4 μg/mL, 3.5 μg/mL, 3.6 μg/mL, 3.7 μg/mL, 3.8 μg/mL, 3.9 μg/mL, 4 μg/mL, 4.1 μg/mL, 4.2 μg/mL, 4.3 μg/mL, 4.4 μg/mL, 4.5 μg/mL, 4.6 μg/mL, 4.7 μg/mL, 4.8 μg/mL, 4.9 μg/mL, 5 μg/mL, 5.1 μg/mL, 5.2 μg/mL, 5.3 μg/mL, 5.4 μg/mL, 5.5 μg/mL, 5.6 μg/mL, 5.7 μg/mL, 5.8 μg/mL, 5.9 μg/mL, 6 μg/mL, 6.1 μg/mL, 6.2 μg/mL, 6.3 μg/mL, 6.4 μg/mL, 6.5 μg/mL, 6.6 μg/mL, 6.7 μg/mL, 6.8 μg/mL, 6.9 μg/mL, 7 μg/mL, 7.1 μg/mL, 7.2 μg/mL, 7.3 μg/mL, 7.4 μg/mL, 7.5 μg/mL, 7.6 μg/mL, 7.7 μg/mL, 7.8 μg/mL, 7.9 μg/mL, 8 μg/mL, 8.1 μg/mL, 8.2 μg/mL, 8.3 μg/mL, 8.4 μg/mL, 8.5 μg/mL, 8.6 μg/mL, 8.7 μg/mL, 8.8 μg/mL, 8.9 μg/mL, 9 μg/mL, 9.1 μg/mL, 9.2 μg/mL, 9.3 μg/mL, 9.4 μg/mL, 9.5 μg/mL, 9.6 μg/mL, 9.7 μg/mL, 9.8 μg/mL, 9.9 μg/mL, 10 μg/mL, a range between any two of these values, or any value between 0.1 μg/mL to 10 μg/mL.

An area under curve (AUC) of a plot of a concentration of the PARP inhibitor (e.g., olaparib or NMS-293) in a blood of the subject over time (e.g., AUC₀₋₂₄ for the first 24 hours after administration) when the PARP inhibitor is administered alone or in combination with the PLK1 inhibitor can be from about 1 μg.h/mL to about 100 μg.h/mL. For example, the AUC of a plot of a concentration of the PARP inhibitor (e.g., olaparib) in a blood of the subject over time (e.g., AUC₀₋₂₄ for the first 24 hours after administration) when the PARP inhibitor is administered alone or in combination with the PLK1 inhibitor can be, or be about, 1 pg.h/mL, 5 pg.h/mL, 10 pg.h/mL, 20 pg.h/mL, 30 pg.h/mL, 40 pg.h/mL, 50 pg.h/mL, 60 pg.h/mL, 70 pg.h/mL, 80 pg.h/mL, 90 pg.h/mL, 100 pg.h/mL, 200 pg.h/mL, 300 pg.h/mL, 400 pg.h/mL, 500 pg.h/mL, 600 pg.h/mL, 700 pg.h/mL, 800 pg.h/mL, 900 pg.h/mL, 1000 pg.h/mL, 2000 pg.h/mL, 3000 pg.h/mL, 4000 pg.h/mL, 5000 pg.h/mL, 6000 pg.h/mL, 7000 pg.h/mL, 8000 pg.h/mL, 9000 pg.h/mL, 10000 pg.h/mL, 50000 pg.h/mL, 100000 pg.h/mL, 500000 pg.h/mL, 1000000 pg.h/mL (1 μg.h/mL), 2 μg.h/mL, 3 μg.h/mL, 4 μg.h/mL, 5 μg.h/mL, 6 μg.h/mL, 7 μg.h/mL, 8 μg.h/mL, 9 μg.h/mL, 10 μg.h/mL, 11 μg.h/mL, 12 μg.h/mL, 13 μg.h/mL, 14 μg.h/mL, 15 μg.h/mL, 16 μg.h/mL, 17 μg.h/mL, 18 μg.h/mL, 19 μg.h/mL, 20 μg.h/mL, 21 μg.h/mL, 22 μg.h/mL, 23 μg.h/mL, 24 μg.h/mL, 25 μg.h/mL, 26 μg.h/mL, 27 μg.h/mL, 28 μg.h/mL, 29 μg.h/mL, 30 μg.h/mL, 31 μg.h/mL, 32 μg.h/mL, 33 μg.h/mL, 34 μg.h/mL, 35 μg.h/mL, 36 μg.h/mL, 37 μg.h/mL, 38 μg.h/mL, 39 μg.h/mL, 40 μg.h/mL, 41 μg.h/mL, 42 μg.h/mL, 43 μg.h/mL, 44 μg.h/mL, 45 μg.h/mL, 46 μg.h/mL, 47 μg.h/mL, 48 μg.h/mL, 49 μg.h/mL, 50 μg.h/mL, 51 μg.h/mL, 52 μg.h/mL, 53 μg.h/mL, 54 μg.h/mL, 55 μg.h/mL, 56 μg.h/mL, 57 μg.h/mL, 58 μg.h/mL, 59 μg.h/mL, 60 μg.h/mL, 61 μg.h/mL, 62 μg.h/mL, 63 μg.h/mL, 64 μg.h/mL, 65 μg.h/mL, 66 μg.h/mL, 67 μg.h/mL, 68 μg.h/mL, 69 μg.h/mL, 70 μg.h/mL, 71 μg.h/mL, 72 μg.h/mL, 73 μg.h/mL, 74 μg.h/mL, 75 μg.h/mL, 76 μg.h/mL, 77 μg.h/mL, 78 μg.h/mL, 79 μg.h/mL, 80 μg.h/mL, 81 μg.h/mL, 82 μg.h/mL, 83 μg.h/mL, 84 μg.h/mL, 85 μg.h/mL, 86 μg.h/mL, 87 μg.h/mL, 88 μg.h/mL, 89 μg.h/mL, 90 μg.h/mL, 91 μg.h/mL, 92 μg.h/mL, 93 μg.h/mL, 94 μg.h/mL, 95 μg.h/mL, 96 μg.h/mL, 97 μg.h/mL, 98 μg.h/mL, 99 μg.h/mL, 100 μg.h/mL, a range between any two of these values, or any value between 10 pg.h/mL and 100 pg.h/mL. For example, the PARP inhibitor is olaparib, and the AUC of a plot of a concentration of olaparib in a blood of the subject over time (e.g., AUC₀₋₁₀ h for the first 10 hours after administration) when olaparib is administered alone or in combination with the PLK1 inhibitor can be, or be about, 0.5 μg.h/mL, 1 μg.h/mL, 1.5 μg.h/mL, 2 μg.h/mL, 2.5 μg.h/mL, 3 μg.h/mL, 3.5 μg.h/mL, 4 μg.h/mL, 4.5 μg.h/mL, 5 μg.h/mL, 5.5 μg.h/mL, 6 μg.h/mL, 6.5 μg.h/mL, 7 μg.h/mL, 8 μg.h/mL, 9 μg.h/mL, 10 μg.h/mL, 20 μg.h/mL, 30 μg.h/mL, 40 μg.h/mL, 50 μg.h/mL, 100 μg.h/mL, or a number or a range between any two of these values.

A time (Tₘₐₓ) to reach a maximum concentration of the PARP inhibitor (e.g., olaparib or NMS-293) in a blood of the subject when the PARP inhibitor is administered alone or in combination with the PLK1 inhibitor can be from about 3 hours to 10 hours. For example, the time (Tₘₐₓ) to reach a maximum concentration of the PARP inhibitor (e.g., olaparib) in a blood of the subject when the PARP inhibitor is administered alone or in combination with the PLK1 inhibitor can be, or be about, 2 hours, 3 hours, 4 hours, 5 hours, 6 hours, 7 hours, 8 hours, 9 hours, 10 hours, 12 hours, 15 hours, 18 hours, 21 hours, 24 hours, a range between any two of these values, or any value between 2 hours and 24 hours. For example, the PARP inhibitor is olaparib, and the time (Tₘₐₓ) to reach a maximum concentration of olaparib in a blood of the subject when olaparib is administered alone or in combination with the PLK1 inhibitor can be, or be about 4 hours, 5 hours, 6 hours, 8 hours, 12 hours, 18 hours, or a number or a range between any two of these values.

An elimination half-life (T_{1/2}) of the PARP inhibitor (e.g., olaparib or NMS-293) in a blood of the subject when the PARP inhibitor is administered alone or in combination with the PLK1 inhibitor can be from about 10 hours to about 100 hours. For example, the elimination half-life (T_{1/2}) of the PARP inhibitor (e.g., olaparib) in a blood of the subject when the PARP inhibitor is administered alone or in combination with the PLK1 inhibitor can be, or be about, 10 hours, 15 hours, 20 hours, 25 hours, 30 hours, 35 hours, 40 hours, 45 hours, 50 hours, 55 hours, 60 hours, 65 hours, 70 hours, 75 hours, 80 hours, 85 hours, 90 hours, 95 hours, 100 hours, a range between any two of these values, or any value between 10 hours and 100 hours. For example, the PARP inhibitor is olaparib, and the elimination half-life (T_{1/2}) of olaparib in a blood of the subject when olaparib is administered alone or in combination with the PLK1 inhibitor can be, or be about, 40 hours, 50 hours, 60 hours, 70 hours, 80 hours, 90 hours, 100 hours, or a number or a range between any two of these values.

The treatment of the present disclosure can comprise administration of a PLK1 inhibitor (onvansertib) for a desired duration in a cycle. The administration of the PLKs inhibitor (and/or the one or more chemotherapeutic agents) can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor (and/or the one or more chemotherapeutic agents) is administered to the patient. The administration can be, for example, once every two days, every three days, every four days, every five days, every six days, or every seven days. The length of the desired duration can vary, for example, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the PLK1 inhibitor (e.g., onvansertib) and/or the one or more chemotherapeutic agents for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of the PLK1 inhibitor (e.g., onvansertib) and/or the one or more chemotherapeutic agents for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) and/or the one or more chemotherapeutic agents in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days.

In some embodiments, the PLK1 inhibitor (e.g., onvansertib) is administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26), In some embodiments, for example when the patient is identified to have low tolerance to the PLK1 inhibitor (e.g., onvansertib), the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor (e.g., onvansertib) is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment. For combination treatment, the administration cycles, dosing schedules, and/or dosage amounts of the PARP inhibitor and the PLK1 inhibitor can be the same or different. For combination treatment, the administration cycle, dosing schedule, and/or dosage amount of the PARP inhibitor can be adjusted according to the administration cycle, dosing schedule, and/or dosage amount of the PLK1 inhibitor. For example, the PARP inhibitor (e.g., olaparib or NMS-293) can be administered in four 7-day cycles (e.g., daily dose on Days 1-5 and no dose on Days 6-7, repeated for 4 weeks), which corresponds to a 28-day cycle for administration of the PLK1 inhibitor (e.g., onvansertib).

The treatment can comprise administration of the PLK1 inhibitor (e.g., onvansertib) at, or at about, 6 mg/m²-90 mg/m², for example, as a daily dose. For example, the treatment can comprise daily administration of the PLK1 inhibitor (e.g., onvansertib) at, or at about, 6 mg/m², 8 mg/m², 10 mg/m², 12 mg/m², 14 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a number or a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK inhibitor (e.g., onvansertib) is administered at 12 mg/m² on twenty days (e.g., Days 1-10 and 15-24) during a 28-day cycle. In some embodiments, the PLK inhibitor (e.g., onvansertib) is administered at 15 mg/m² on ten days (e.g., Days 1-5 and 15-19) during a 28-day cycle. In some embodiments, the PLK inhibitor (e.g., onvansertib) is administered at 8 mg/m² or 10 mg/m² everyday (e.g., Days 11-28) during a 28-day cycle. In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK1 inhibitor is administered at or at about 12 mg/m². In some embodiments, the PLK1 inhibitor is administered at or at about 15 mg/m². In some embodiments, the PLK1 inhibitor is administered at or at about 18 mg/m².

A maximum concentration (Cₘₐₓ) of the PLK1 inhibitor (e.g., onvansertib) in a blood of the subject (during the treatment or after the treatment) when the PLK1 inhibitor is administered alone or in combination with the PARP inhibitor can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the PARP inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvanserib) in a blood of the subject over time (e.g., AUC₀₋₂₄ for the first 24 hours after administration) when the PLK1 inhibitor is administered alone or in combination with the PARP inhibitor can be from about 1000 nmol/L.hour to about 400000 nmol/L.hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the PARP inhibitor can be, or be about, 1000 nmol/L.hour, 5000 nmol/L.hour, 10000 nmol/L.hour, 15000 nmol/L.hour, 20000 nmol/L.hour, 25000 nmol/L.hour, 30000 nmol/L.hour, 35000 nmol/L.hour, 40000 nmol/L.hour, a range between any two of these values, or any value between 1000 nmol/L.hour and 400000 nmol/L.hour.

A time (Tₘₐₓ) to reach a maximum concentration of the PLK1 inhibitor (e.g., onvansertib) in a blood of the subject when the PLK1 inhibitor is administered alone or in combination with the PARP inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the PARP inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the PARP inhibitor can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the PARP inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Additional Cancer Therapeutics or Therapy

Methods, compositions and kits disclosed herein can be used for treating cancer. In some embodiments, a method for treating cancer comprises administrating a PARP inhibitor and a PLK1 inhibitor (e.g., onvansertib) to a subject (e.g., a patient) in need thereof. The method can comprise administering a therapeutically effective amount of the PARP inhibitor and a therapeutically effective amount of the PLK1 inhibitor. The treatment can comprise administration of at least one additional cancer therapeutics or cancer therapy. The treatment can comprise administration a therapeutically effective amount of at least one additional cancer therapeutics or cancer therapy. The PARP inhibitor and the cancer therapeutics or cancer therapy can, for example, co-administered simultaneously or sequentially. The PLK1 inhibitor (e.g., onvansertib) and the cancer therapeutics or cancer therapy can, for example, co-administered simultaneously or sequentially. In some embodiments, the additional cancer therapeutics is cytarabine, low-dose cytarabine (LDAC) and/or decitabine. The safety, pharmacokinetics, and preliminary clinical activity of onvansertib in combination with either LDAC or decitabine have been determined in patients with R/R AML and are described in PCT Application published as WO2021146322, the content of which is incorporated herein by reference in its entirety. In some embodiments, the treatment comprises administration of LDAC at, or at about, 20 mg/m² subcutaneous (SC) once a day (qd) for seven, eight, night, ten, eleven, twelve, or thirteen days in a cycle. In some embodiments, the treatment comprises administration of decitabine at, or at about, 20 mg/m² intravenous (IV) qd for three, four, five, six, or seven days in a cycle. In some embodiments, the treatment comprises administration of LDAC at, or at about, 20 mg/m² subcutaneous (SC) once a day (qd) for ten days in a cycle, and administration of decitabine at 20 mg/m² intravenous (IV) qd for five days in a cycle.

### Methods for Predicting/Determining Treatment Efficacy and Status for Cancer

Also disclosed herein include methods, compositions, kits, and systems for predicting/determining clinical outcome for a combination treatment of cancer of the present disclosure, monitoring of the combination treatment, predicting/determining responsiveness of a subject to the combination treatment, determining the status of the cancer in a subject, and improving combination treatment outcome. The methods, compositions, kits and systems can be used to guide the combination treatment, provide combination treatment recommendations, reduce or avoid unnecessary ineffective combination treatment for patients. ctDNA can be analyzed to predict/determine clinical outcome for cancer treatment using a combination of a PARP inhibitor and a PLK1 inhibitor of the present disclosure, monitor the combination treatment, predict/determine responsiveness of a subject to the combination treatment, determine cancer status in a subject, improve combination treatment outcome, guide combination treatment, provide combination treatment recommendations, and/or to reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application published as WO2021146322, the content of which is incorporated herein by reference in its entirety.

A method of determining responsiveness of a subject to a combination treatment comprising a PARP inhibitor and a PLK1 inhibitor of the disclosure can comprise, for example, analyzing circulating tumor DNA (ctDNA) of a subject with cancer, the subject is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

In some embodiments, the first time point is prior or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining cancer status of a subject, comprising analyzing circulating tumor DNA (ctDNA) of a subject, thereby determining cancer status of the subject. The subject can be a subject undergoing a current combination treatment comprising a PARP inhibitor and a PLK1 inhibitor of the present disclosure, a subject that has received a prior combination treatment of the present disclosure, and/or a subject that is in remission for the cancer. The subject in remission for cancer can be in complete remission (CR), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of cancer relapse or is in cancer relapse.

In some embodiments, the first time point is prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

In some embodiments, the method comprises starting an additional treatment to the subject if the subject is indicated as in cancer relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is MAF for one or more driver mutations of the cancer (e.g., ovarian cancer, breast cancer, prostate cancer, colorectal cancer, pancreatic cancer, or a combination thereof). In some embodiments, Log₂(C₁/C₀)< a MAF threshold indicates a decrease in ctDNA MAF Co is ctDNA MAF in the first sample and C₁ is ctDNA MAF in one of the additional samples. In some embodiments, the MAF threshold is, or is about, 0.01 to-0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in in the 75 genes. In some embodiments, one or more the driver mutations are mutations in the 75 genes.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for the cancer. The method can comprise: detecting variant allele frequency in circulating tumor DNA (ctDNA) obtained from a subject at a first time point in a first sample before the subject undergoes a combination treatment comprising a PARP inhibitor and a PLK1 inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples, a decrease in the variant allele frequency in at least one of the additional samples relative to the first sample indicates the subject as responsive to the combination treatment; and continuing the combination treatment to the subject if the subject is indicated as responsive to the combination treatment, or discontinuing the combination treatment to the subject and/or starting a different cancer treatment to the subject if the subject is not indicated as responsive to the combination treatment.

Also disclosed herein include methods of treating cancer The method can comprise: administering a combination treatment comprising a PARP inhibitor and a PLK1 inhibitor of the present disclosure to a subject in need thereof; determining a decrease, relative to a variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with cancer, for example a subject that has not received any prior cancer treatment before the combination treatment. In some embodiments, the subject has received prior cancer treatment and was in remission for the cancer, for example a subject in complete remission (CR), or in partial remission (PR) after receiving the prior combination treatment.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is mutant allelic frequency (MAF) for one or more driver mutations of the cancer (e.g., ovarian cancer, breast cancer, prostate cancer, colorectal cancer, pancreatic cancer, or a combination thereof). In some embodiments, Log₂(C₁/C₀)< a MAF threshold indicates a decrease in ctDNA MAF C₀ is ctDNA MAF in the first sample and C₁ is ctDNA MAF in one of the additional samples. In some embodiments, the MAF threshold is-0.05.

The driver mutation can be, for example, a mutation in one of the 75 genes set forth in Table 3, at least one of the one or more the driver mutations is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2, and/or one or more the driver mutations are mutations in the 75 genes. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the cancer. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

### Compositions and Kits

Disclosed herein include compositions and kits for treating cancer. In some embodiments, a kit comprises: a Polo-like kinase 1 (PLK1) inhibitor; and a manual providing instructions for co-administrating the PLK1 inhibitor with a PARP inhibitor to a subject for treating cancer. In some embodiments, the kit comprises the PARP inhibitor. The cancer can be, for example, ovarian cancer, breast cancer, prostate cancer, colorectal cancer, pancreatic cancer, or a combination thereof.

In some embodiments, the subject has cancer (e.g., ovarian cancer, breast cancer, prostate cancer, colorectal cancer, pancreatic cancer, or a combination thereof). In some embodiments, the instructions comprise instructions for co-administrating the PLK inhibitor and the PARP inhibitor simultaneously. In some embodiments, the instructions comprise instructions for co-administrating the PLK inhibitor and the PARP inhibitor sequentially. In some embodiments, the instructions comprise instructions for administering of the PLK1 inhibitor orally. In some embodiments, the instructions comprise instructions for administrating the PARP inhibitor orally.

In some embodiments, the instructions comprise instructions the subject has received a prior PARP inhibitor treatment. In some embodiments, the instructions comprise instructions the subject did not respond to treatment with the PARP inhibitor alone. In some embodiments, the instructions comprise instructions the subject is known to be resistant to a PARP inhibitor therapy.

In some embodiments, the instructions comprise instructions the subject has received at least one prior treatment for the cancer. In some embodiments, the prior treatment does not comprise the use of a PARP inhibitor, a PLK inhibitor, or both. In some embodiments, the instructions comprise instructions the subject was in remission for the cancer. In some embodiments, the subject in remission for cancer was in complete remission (CR), or in partial remission (PR).

In some embodiments, the instructions comprise instructions for administering each of the PARP inhibitor and the PLK1 inhibitor to the subject in a cycle of at least twice within a week. In some embodiments, the instructions comprise instructions for administering each of the PARP inhibitor and the PLK1 inhibitor to the subject in a cycle of at least five times within a week In some embodiments, the instructions comprise instructions for administering the PARP inhibitor, the PLK1 inhibitor, or both are in a cycle of at least 7 days. In some embodiments, each cycle of treatment is at least about 21 days. In some embodiments, each cycle of treatment is from about 21 days to about 28 days, for example 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the PARP inhibitor daily. In some embodiments, the instructions comprise instructions for administrating the PARP inhibitor and the PLK1 inhibitor for at least two cycles.

In some embodiments, the PARP inhibitor is selective and/or specific for PARP inhibition (e.g., PARP1 inhibitor, PARP2 inhibition, or both). In some embodiments, the PARP inhibitor is iniparib (BSI 201), talazoparib (BMN-673), AZD5305, olaparib (AZD-2281), rucaparib (AG014699, PF-01367338), ABT-888, veliparib (ABT-888), niraparib, CEP 9722, MK 4827, BGB-290 (pamiparib), BSI-201, CEP-8983, E7016, 3-aminobenzamide, or a combination thereof. In some embodiments, the PARP inhibitor is olaparib. In some embodiments, the PARP inhibitor is or NMS-293.

In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (B1I6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib. In some embodiments, the PARP inhibitor is olaparib, and the PLK1 inhibitor is onvansertib.

In some embodiments, the instructions comprise instructions for administering the PLK1 inhibitor at 12 mg/m²-90 mg/m². In some embodiments, the instructions comprise instructions for administering the PARP inhibitor at 20 mg−1200 mg.

The methods, compositions and kits disclosed herein can also be used to sensitize cancer cells to one or more PARP inhibitors. The method can comprise contacting cancer cells with a composition comprising a PLK1 inhibitor (e.g., onvansertib), or a pharmaceutically acceptable salt, solvate, stereoisomer, or prodrug thereof, thereby sensitizing the cancer cells to the one or more PARP inhibitors (e.g., olaparib or NMS-293). Contacting cancer cells with the composition can occur in vitro, ex vivo, in vivo, or in any combination. In some embodiments, contacting cancer cells with the composition is in a subject's body. In some embodiments, cancer cells are contacted with the composition in a cell culture. The subject can be a mammal, for example a human. The sensitization of the cancer cells can increase the responsiveness of the cancer cells to the one or more PARP inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the cancer cells to the one or more PARP inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the cancer cells is, in some embodiments, relative to the untreated cancer cells. The sensitization of the cancer cells can increase the responsiveness of the subject having the cancer cells to one or more PARP inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the subject having the cancer cells to the one or more PARP inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the subject having the cancer cells is, in some embodiments, relative to the subjects untreated with the composition.

The method can comprise determining sensitization of the cancer cells to the one or more PARP inhibitors after being contacted with the PLK1 inhibitor. The method can comprise contacting the cancer cells with the one or more PARP inhibitors concurrently and/or after being contacted with the PLK1 inhibitor. In some embodiments, contacting the cancer cells with the one or more PARP inhibitors occurs in the body of a subject. The subject can be a mammal, for example human. The subject can be, for example, a subject that did not respond to, or is known to be resistant to, PARP inhibitors alone. The subject can be, for example, a subject that had prior treatment with one of the one or more PARP inhibitors. In some embodiments, the method comprises determining the response of the subject to the one or more PARP inhibitors.

### EXAMPLES

Some aspects of the embodiments discussed above are disclosed in further detail in the following example, which are not in any way intended to limit the scope of the present disclosure.

### Example 1

### Determining Antitumor Activity of Olaparib and Onvansertib in BRCA1 Deficient Models

PLK1 inhibitors as single agents can be of therapeutical value in BRCA deficient tumors (triple negative, BRCA1-2 mutated breast and ovarian). In this example, the efficacy of onvansertib in combination with olaparib, a PARP inhibitor, was evaluated.

### Step 1. Toxicological Study

Immuno-competent mice were treated orally with a combination of 75 mg/kg olaparib and 40 mg/kg onvansertib per 5 days a week for two weeks, or (3) with a combination of 100 mg/kg olaparib and 40 mg/kg onvansertib per 5 days a week for two weeks.

Mice (5 animals per group) were weighted twice a week and clinical examination of the mice was done daily. Mice were then observed for another 1 week and if no toxicity was noticed, the dose of onvansertib was scaled up to 50 mg/kg and mice treated for other two weeks.

The combination of onvansertib (40 or 50 mg/kg) and Olaparib (75 and 100 mg/kg) was tolerated by the treated mice (no body weight loss>20%).

### Step 2. Evaluation of the Antitumor Activity of the Combination

The combination of onvansertib and olaparib was tested in two BRCA1-mutant high grade serous ovarian cancer (HGSOC) PDX (patient-derived xenograft) models resistance to olaparib: PDX #HOC22 and PDX #HOC266. Their main characteristics are summarized in Table 1. Enclosed are the characteristics of both BRCA1 and TP53 status, and platinum and olaparib tumor responsiveness.

TABLE 1
Molecular and pharmacological characteristics of the BRCA1-mutant PDXs

| | Platinum | Olaparib | | | Tumor |
|---|---|---|---|---|---|
| PDX ID# | sensitivity | sensitivity | TP53 status | BRCA1 status | growth |
| PDX# HOC22 | +++ | − | mut | mut | ip |
| PDX# HOC266 | +++ | − | mut | mut | ip |

++++: very sensitive;
−: resistant;
i.p.: intraperitoneally transplanted;
mut = mutant

Mice were transplanted ip with 5×10⁶ and were randomized to receive the given treatments (as described below) two weeks after tumor transplantation.

Mice (8 mice/group) were treated for 4 weeks with the following: (1) vehicle; (2) olaparib (OLA) for 5 days a week for 4 weeks. In one example, 100 mg/kg was given to PDX#HOC22 and 75 mg/kg to PDX #HOC266; (3) onvansertib (ONVA) (50 mg/kg) for 5 days a week for 4 weeks; or (4) the combination of OLA+ONVA.

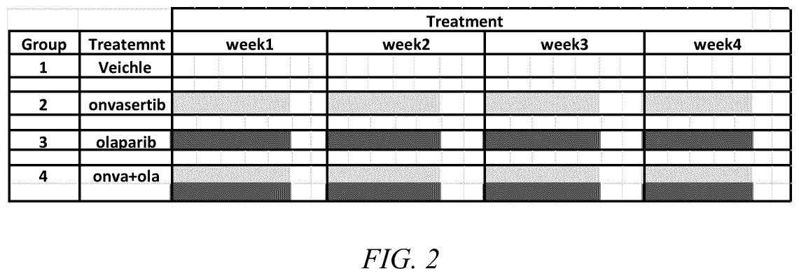

Onvansertib were administrated 2 hours prior to olaparib on the days that both onvansertib and olaparib were administered to the mice (see FIG. 2).

An additional 8 mice per group were transplanted for pharmacodynamic studies. Four weeks after tumor transplant, mice were treated for one week as described above and sacrificed at two hours and 24 hours after the last olaparib dose.

Ascites were collected, cells were recovered, counted and part of cells were snap frozen (for protein, DNA and RNA extraction) and part were paraffin embedded. IHC Ki67, Rad51 foci expression were measured. By western blot, gammaH2AX (markers of both DNA damage and apoptosis induction) and the activation of caspases (a read out of apoptosis induction) were measured. As the samples were available, additional markers were assessed.

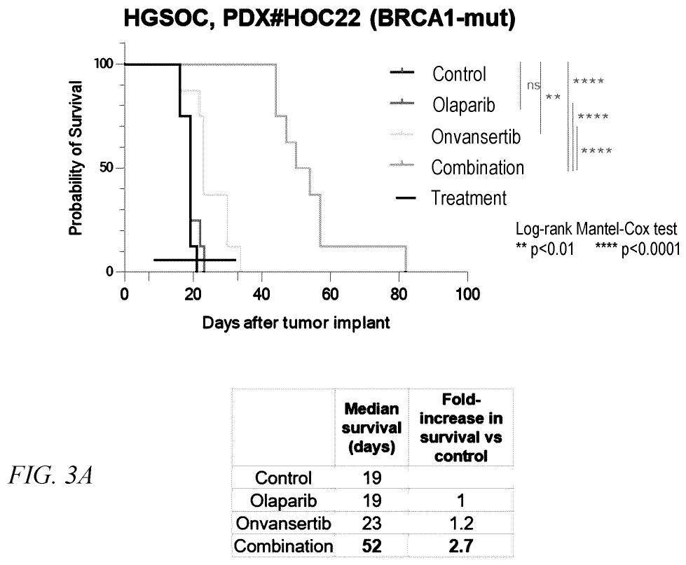

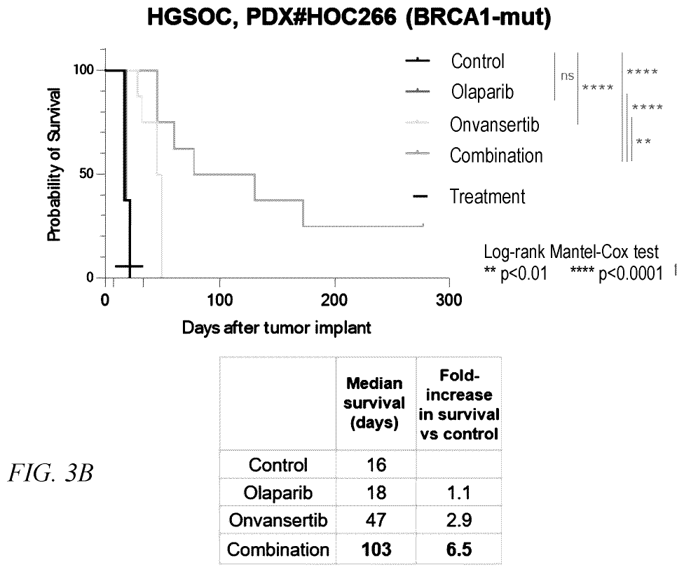

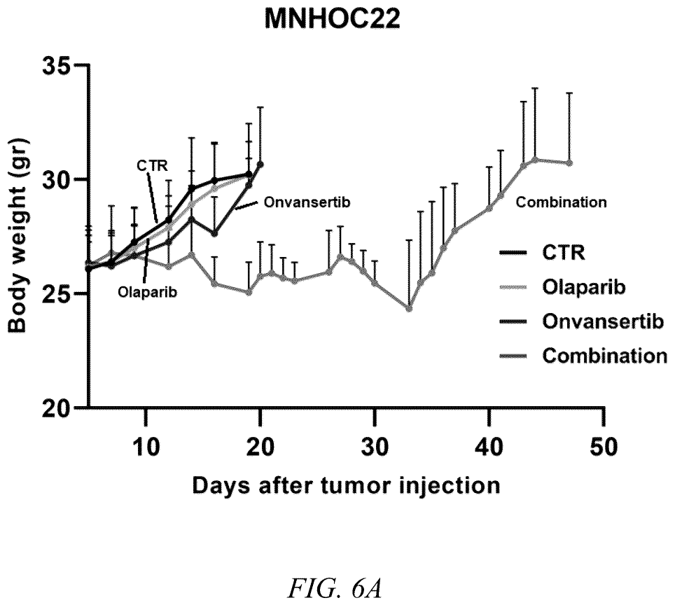

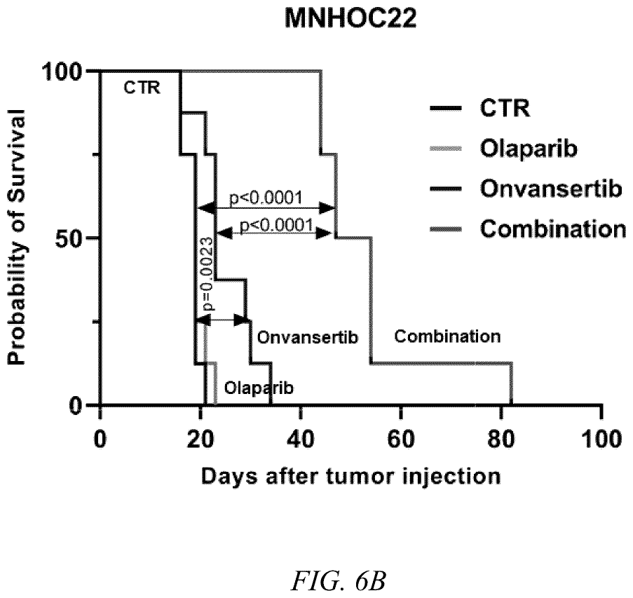

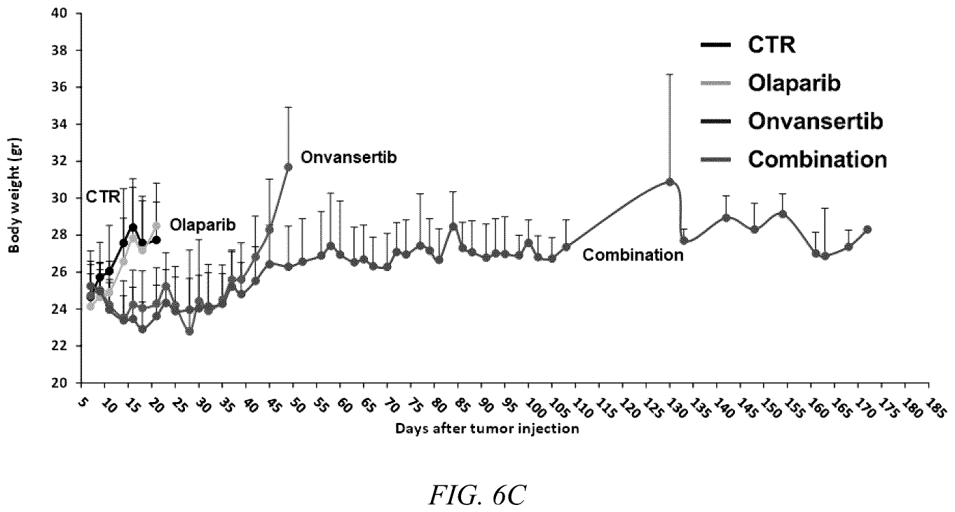

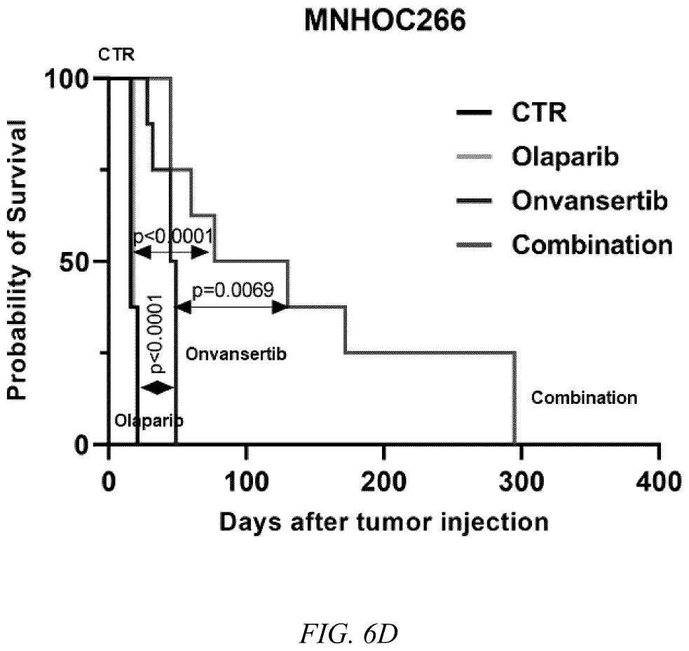

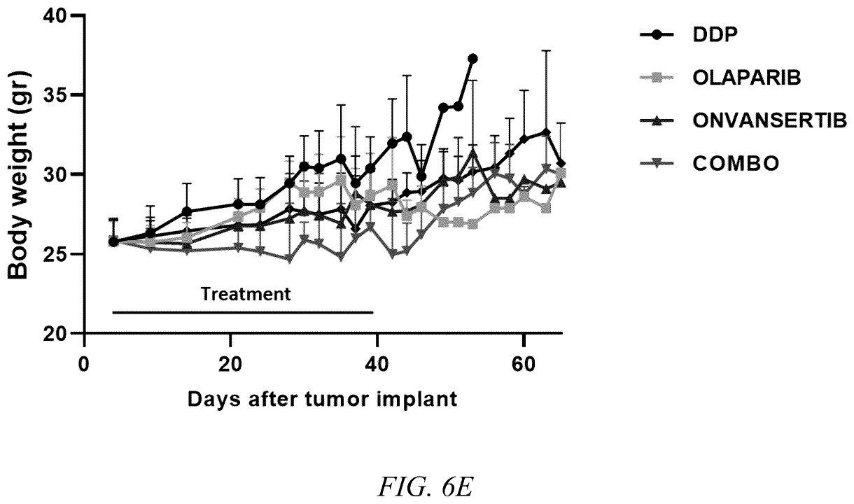

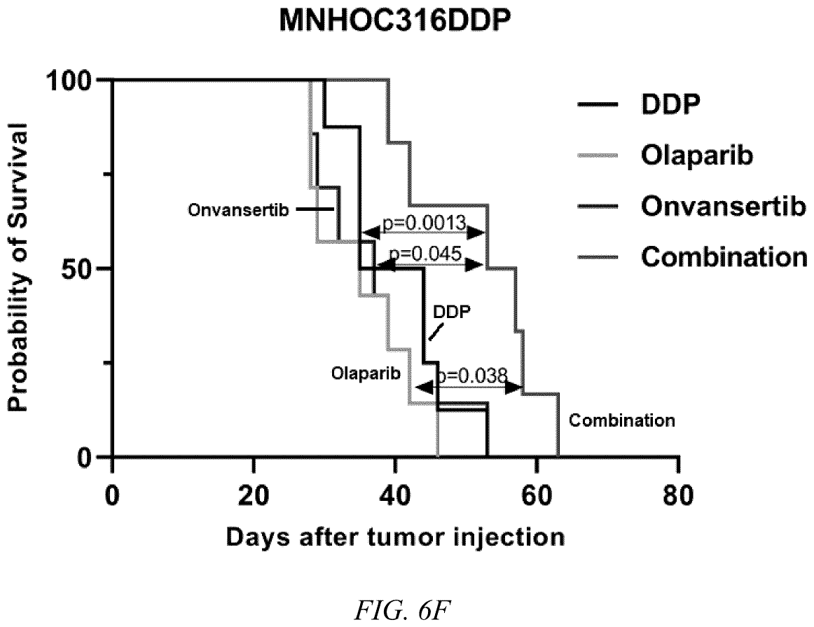

FIG. 3A is a Kaplan Meier curve showing the probably of survival in the ovarian BRCA1-mutant PDX #HOC22 model and FIG. 3B is a Kaplan Meier curve showing the probability of survival in the ovarian BRCA1-mutant PDX #HOC266 model. The data indicates that in the two BRCA1-mutated olaparib-resistant PDX models, onvansertib in combination with olaparib significantly increases mice survival in comparison with vehicle treatment and single agent treatments (olaparib or onvansertib). In particular, in the PDX #HOC266 model, 2 out of 8 mice in the combination group were still alive at day 300 after tumor implant. The mice were sacrificed at Day 300 and 1 mouse was tumor-free (FIG. 6D). Without being bound to any particular theory, it is believed that onvansertib as an PLK1 inhibitor sensitized tumor cells to PARP inhibitor such as olaparib to achieve effective cancer treatment.

### Example 2

### Determining Antitumor Activity of Olaparib and Onvansertib in BRCA1 Deficient Models

The combination of onvansertib and olaparib are tested using similar protocol described in Example 1 in two PDX models resistant to olaparib: PDX #218ola and PDX #154. Their main characteristics are summarized in Table 2. PDX #218ola is a PDX derived from PDX #218 highly sensitive to olaparib by different in vivo cycles of olaparib treatment until olaparib treatment was no longer effective.

TABLE 2
Molecular and pharmacological characteristics of the PDXs

| | Platinum | Olaparib | | | Tumor |
|---|---|---|---|---|---|
| PDX ID# | sensitivity | sensitivity | TP53 status | BRCA1 status | growth |
| PDX# 218ola | +++ | − | mut | mut | sc |
| PDX# 154 | ++ | − | mut | mut | sc |

++++: very sensitive
−: resistant;
i.p.: intraperitoneally transplanted
s.c. subcutaneous transplanted

Mice are transplanted s.c. with PDX #218ola and PDX #154 tumor fragments (3 mm×3 mm) and are randomized to receive the treatments as described below when tumor masses reach 100-150 mm³.

Mice (10 mice/group) are treated for 4 to 5 weeks with the following: (1) vehicle; (2) Olaparib (OLA), 80 mg/kg for 5 days a week for 4 to 5 weeks; (3) Onvansertib (ONVA) 45 mg/kg for 5 days a week for 4 to 5 weeks; (4) ONVA 30 mg/kg for 5 days a week for 4 to 5 weeks; (5) the combination of OLA 80 mg/kg+ONVA 45 mg/kg for 5 days a week for 4-5 weeks; or (6) the combination of OLA 80 mg/kg+ONVA 30 mg/kg for 5 days a week for 4-5 weeks

Onvansertib were administrated 2 hours prior to olaparib.

For pharmacodynamic experiments, mice are transplanted and treated for 5 days as described above and sacrificed at the following timepoints:

1-vehicle: at 4 hr (n=4) and 24 hr (n=4) after the last treatment (total=8)
2-OLA: at 2 hr (n=4) and 22 hr (n=4) after the last dose of olaparib (total=8)
3-ONVA 45 mg/kg: at 4 hr (n=4), 8 hr (n=3) and 24 hr (n=4) after the last dose of onvansertib (total=11)
4-ONVA 30 mg/kg: at 4 hr (n=4), 8 hr (n=3) and 24 hr (n=4) after the last dose of onvansertib (total=11)
5-the combination of OLA 80 mg/kg+ONVA 45 mg/kg: at 4 hr (n=4), 8 hr (n=3) and 24 hr (n=4) after the last dose of onvansertib (total=11)
6-the combination of OLA 80 mg/kg+ONVA 30 mg/kg: at 4 hr (n=4), 8 hr (n=3) and 24 hr (n=4) after the last dose of onvansertib (total=11)

Blood are collected for pharmacokinetics analysis from all mice at the same timepoints as the tumors are collected. At 4 hr and 24 hr, tumors can be collected and divided in two parts: one part can be snaped frozen for protein, DNA and RNA extraction and the other part can be paraffin embedded. IHC Ki67can be measured. By western blot, gammaH2aX, the activation of caspases, and phospho H3 (read out of M2 block) can be measured. In addition, activation of the DNA damage response pathway can be evaluated by ATR/Chk1 axis.

### Example 3

### Antitumor Activity of Olaparib and Onvansertib in BRCA1 Wildtype Models

In this example, the efficacies of onvansertib alone and in combination with olaparib were evaluated in BRCA1 wildtype models. The same protocol of Example 1 was used in this example unless noted otherwise.

The combination of onvansertib and olaparib was tested in three BRCA1-WT HGSOC PDX models resistant to olaparib: PDX #124, PDX #239, and PDX #76. Their main characteristics are summarized in Table 3.

TABLE 3
Molecular and pharmacological characteristics
of the BRCA1-WT PDXs

| | Platinum | Olaparib | TP53 | BRCA1 | Tumor |
|---|---|---|---|---|---|
| PDX ID# | sensitivity | sensitivity | status | status | growth |
| PDX# 124 | +++ | − | mut | wt | sc |
| PDX# 239 | +++ | − | mut | wt | sc |
| PDX# 76 | − | − | mut | wt | i.p |

++++: very sensitive;
−: resistant;
i.p.: intraperitoneally transplanted,
s.c. subcutaneously transplanted

Mice are transplanted with tumor fragment and randomized to receive treatment. For example, mice can be transplanted s.c. with 3 mm³ tumor fragment or i.p. with 5×10⁶ cells and then randomized to receive treatment two weeks after tumor transplantation or when tumor reaches 100-150 mm³ size. In the PDX #124 model, mice were transplanted s.c. with 3 mm³ tumor fragment and randomized to receive treatment when tumor reached 100-150 mm³ size.

Mice (8 mice/group) were treated for 4 weeks with the following: (1) vehicle; (2) Onvansertib (ONVA) (50 mg/kg) for 5 days a week; (3) Olaparib (OLA) (100 mg/kg) for 5 days a week; or (4) the combination of OLA (100 mg/kg)+ONVA (50 mg/kg) for 5 days a week.

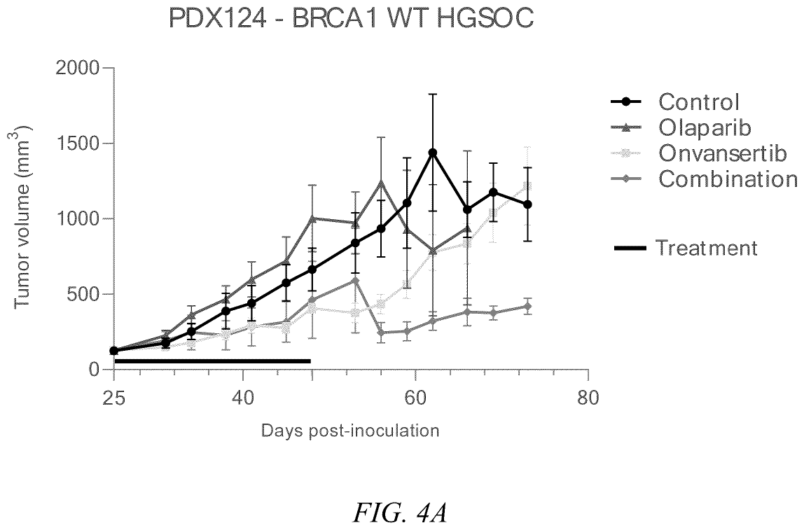

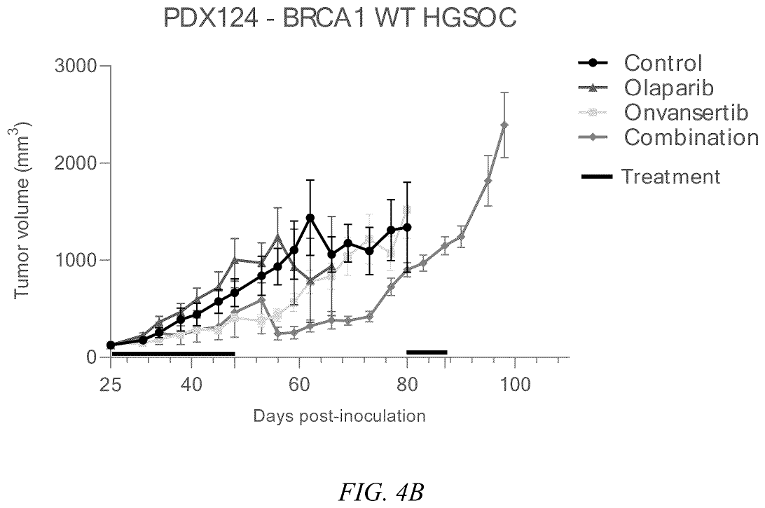

FIG. 4A is a plot showing tumor volume in a BRCA1-WT HGSOC PDX model (PDX #124). The combination of onvansertib and olaparib showed significant tumor growth inhibition in comparison with the control (Kruskal-Wallis test, p=0.04), while the single agent (onvansertib or olaparib) did not show significant anti-tumor activity.

For the combination group, treatment was resumed on Day 80 for one week. FIG. 4B shows tumor volume in the same PDX #124 model with the resumed treatment in the combination group. The data indicates that resuming treatment with the combination of onvansertib and olaparib after 32 days of drug holiday slowed down tumor growth, supporting that the combination can be efficacious with drug holiday schedules.

### Example 4

### Antitumor Activity of Olaparib and Onvansertib in BRCA1 Deficient and Wildtype Models

In this example, the efficacies of onvansertib alone and in combination with olaparib were evaluated in three HGSOC models. The same protocol of Example 1 was used in this example unless noted otherwise.

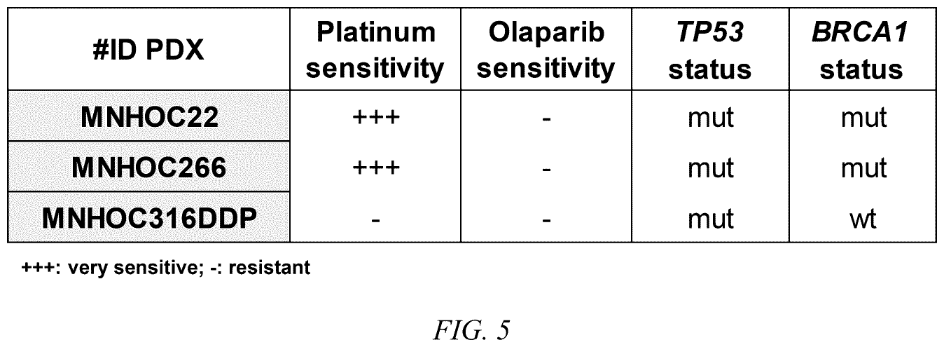

The patient-derived xenografts (PDXs) used in this example are part of a human ovarian xenobank recently established at the Mario Negri Institute in Milan (IT) and described in Ricci F. et al., Cancer Res. 2014, the content of which is incorporated herein by reference. Three models whose molecular and pharmacological characteristics are reported in FIG. 5 were selected. MNHOC316DDP derived from a cisplatin(DDP)-sensitive PDX made resistant through multiple in vivo DDP treatment cycles.

The selected PDXs were orthotopically transplanted in NCr-nu/nu mice and randomized into: 1) Control/vehicle-treated group; 2) Olaparib (100 mg/kg-MNHOC22 and MNHOC316DDP- or 80 mg/kg-MNHOC266-per os); 3) Onvansertib (50 mg/kg, per os); 4) Combination (Combo), 5 days/week for 4 weeks. For MNHOC316DDP, DDP treated mice (5 mg/kg q7×3) were considered as control. The antitumor activity was evaluated by calculating the increase in life span (ILS %)=[(median survival control group-median survival treated group)−median survival treated group]×100).

To perform pharmacodynamic (PD) studies, MNHOC22 and MNHOC266 bearing mice were treated with the doses previously reported for four consecutive days, and then euthanized at 2 hrs and 24 hrs after the last treatment. Ascitic cells were both formalin-fixed paraffin-embedded (FFPE) and snaped frozen for PD studies. PD studies included: proliferation measured by Ki67 IHC stain, apoptosis measured by the Caspase-Glo® 3/7 kit (Promega), mitosis quantified by mitotic events count on FFPE and anti-pH3-Ser10expression by WB, DNA damage/apoptosis quantified by WB using an anti-γH2AX antibody. RAD51-foci were quantified by using an IF-based method as described in Guffanti F. et al., BJC 2022 and by scoring in blind the percentage of RAD51/geminin (GMN)-positive tumor cells with 5 or more foci per nucleus (RAD51+/GMN+). At least 100 GMN-positive cells in three different areas of the tissue section were analysed. Pre-defined threshold was used to determine qualitative scores: RAD51 positive tumors were >10% RAD51+/GMN+cells.

For survival analyses, Kaplan-Mayer curves are reported and Mantel-Cox test was used; unpaired-t test was performed for all the other comparisons. p-value<0.05 was considered significant.

FIGS. 6A, 6C and 6E are plots showing changes in mice body weight in the three PDX models shown in FIG. 5 (MNHOC22, MNHOC26, and MNHOC316DDP, respectively) treated with control, olaparib, onvansertib, or onvansertib and olaparib combination. The data demonstrates that olaparib/onvansertib combination was well tolerated in vivo in all three models; even if a decrease in body weight was observed, it never exceeded 20% and reverted upon drugs withdrawal.

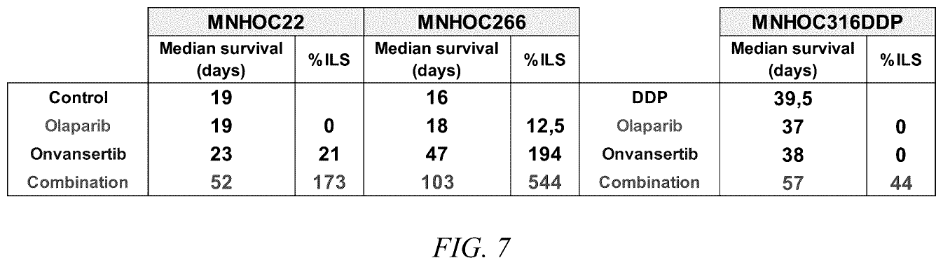

FIGS. 6B, 6D and 6F are Kaplan Meier curves showing the probability of survival in the three PDX models (MNHOC22, MNHOC26, and MNHOC316DDP, respectively) treated with control, olaparib, onvansertib, or onvansertib and olaparib combination. FIG. 7 shows median survival times and increase in life span (ILS) in the three PDXs (MNHOC22, MNHOC266 and MNHOC316DDP). All three PDXs were resistant to olaparib; onvansertib was slightly active in MNHOC266 model, but had no activity in both MNHOC22 and MNHOC316DDP. Conversely, the combination was highly effective as demonstrated by a significant increased survival as compared to controls and single agent treatments.

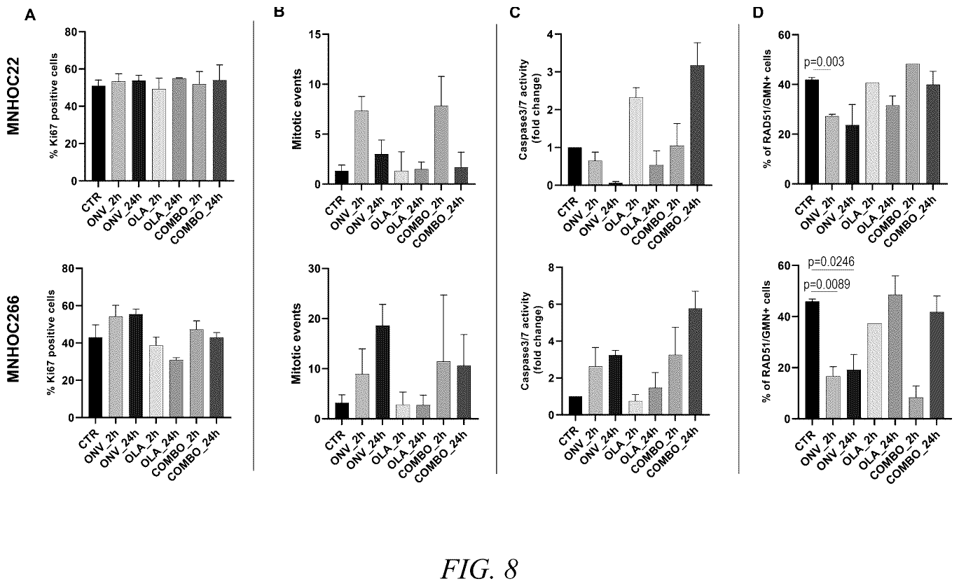

FIG. 8 shows evaluation of Ki67 positivity (panel A), mitosis (panel B), apoptosis (panel C) and RAD51-foci formation (panel D) in PDXs MNHOC22 and MNHOC266. No differences in the % of Ki67 positive cells among the different groups were observed (FIG. 8, panel A); onvansertib treatment induced an increase in mitotic events at 2 hrs both as single agent and in combination with olaparib (FIG. 8, panel B); a higher apoptosis could be seen in both models at 24 hrs (FIG. 8, panel C). A high basal level of RAD51-foci positive cells was observed in both models. A trend toward a decrease in RAD51-foci positive cells after onvansertib treatment could be observed (FIG. 8, panel D).

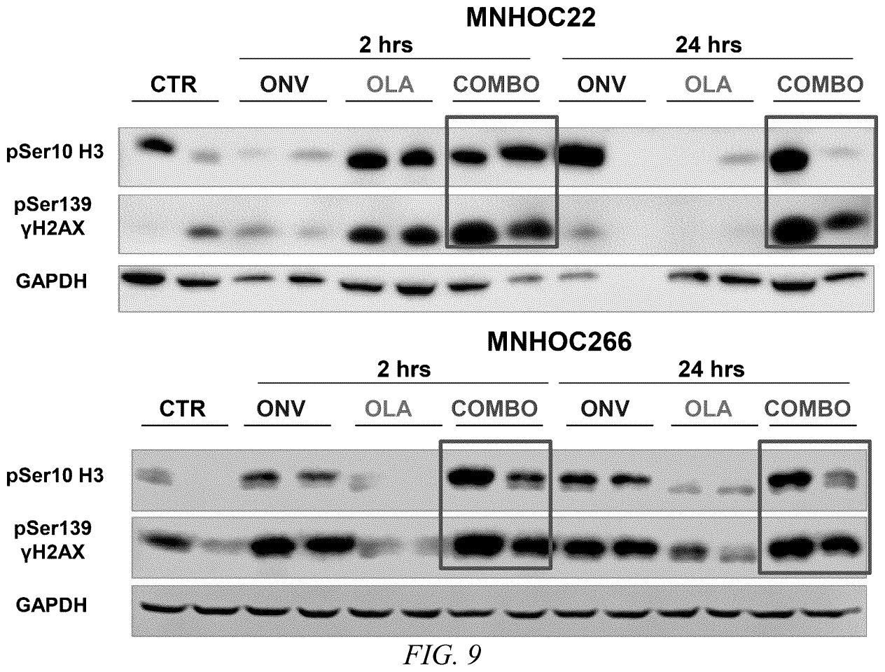

FIG. 9 shows levels of pSer10-H3 and pSer139-γH2AX in tumors of PDXs MNHOC22 and MNHOC266 treated with control, olaparib, onvansertib, or onvansertib and olaparib combination. Higher levels of pSer10-H3 and pSer139-γH2AX were observed in the combination group at both 2 and 24 hrs in both models (FIG. 9), suggesting an increased G2/M block and apoptotic death/DNA damage, corroborating the data on mitotic counts and caspase 3/7 activities (FIGS. 8, panels B and C).

The results show a strong therapeutic efficacy of the olaparib/onvansertib combination in olaparib resistant ovarian carcinoma PDXs. The combination can induce higher G2/M block and apoptosis/DNA damage.

### Example 5

### PLK1 Inhibition Sensitizes Cancer Cells to PARP Inhibitors

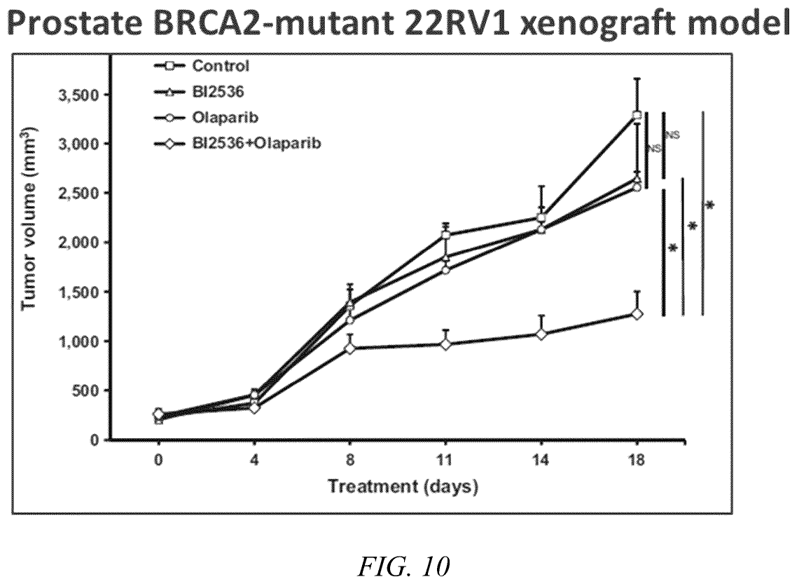

PLK1 inhibition sensitized cells to genotoxic stresses (i.e., radiation) and to PARP inhibitors through impairment of HR in in vitro preclinical studies. PLK1 inhibition also sensitizes tumor cells to PARP inhibition in vivo. For example, the combination of the PLK1 inhibitor BI2536 and the PARP inhibitor olaparib synergistically inhibits growth of prostate BRCA2-mutant xenograft tumors (FIG. 10). As shown herein (e.g., FIGS. 3A-B), in ovarian BRCA1-mutant PDX models resistant to olaparib, the combination of onvansertib and olaparib significantly increased the survival of mice (2.7-fold and 6.5-fold vs control or olaparib single agent).

As shown in this example, onvansertib can sensitize tumors resistant to PARP inhibitors.

### Example 6

### Onvansertib and Olaparib Cell Line Screen

In this example, the synergy between onvansertib and olaparib is tested in vitro on breast, ovarian, pancreatic, and prostate cancer cell lines. Cell lines tested in this example are listed in Table 4. Table 5 provides a list of cell lines with BRCA mutations.

TABLE 4
Cell lines and tissue types

| Cell Line | Tissue Type | Cell Line | Tissue Type |
|---|---|---|---|
| 22RV1 | Prostate | JHOC-5 | Ovary |
| A2780 | Ovary | JHOM-1 | Ovary |
| AsPC-1 | Pancreas | JHOS-2 | Ovary |
| AU565 | Breast | KLM-1 | Pancreas |
| BT-20 | Breast | KP-2 | Pancreas |
| BT-474 | Breast | KP-3 | Pancreas |
| BT-549 | Breast | KP-4 | Pancreas |
| BxPC-3 | Pancreas | KURAMOCHI | Ovary |
| CAL-120 | Breast | MCAS | Ovary |
| CAMA-1 | Breast | MCF7 | Breast |
| Caov-3 | Ovary | MDA-MB-175-VII | Breast |
| CAPAN-2 | Pancreas | MDA-MB-231 | Breast |
| CFPAC-1 | Pancreas | MDA-MB-436 | Breast |
| COV362 | Ovary | MDA-MB-453 | Breast |
| COV434 | Ovary | MDA-MB-468 | Breast |
| COV504 | Ovary | MIA PaCa-2 | Pancreas |
| COV644 | Ovary | OC-316 | Ovary |
| DU-145 | Prostate | OV7 | Ovary |
| DU-4475 | Breast | OV90 | Ovary |
| HCC1954 | Breast | OVCAR-3 | Ovary |
| HCC38 | Breast | OVISE | Ovary |
| HCC70 | Breast | OVK18 | Ovary |
| HMC-1-8 | Breast | OVSAHO | Ovary |
| HPAF-II | Pancreas | OVTOKO | Ovary |
| Panc 04.03 | Pancreas | SUIT-2 | Pancreas |
| PC-3 | Prostate | SUM159PT | Breast |
| PSN1 | Pancreas | T47D | Breast |
| RMUG-S | Ovary | TOV-21G | Ovary |
| SK-BR-3 | Breast | ZR-75-1 | Breast |
| SK-OV-3 | Ovary | ZR-75-30 | Breast |
| SNU-119 | Ovary | SUIT-2 | Pancreas |
| SNU-324 | Pancreas | | |

TABLE 5
Cell lines with BRAC mutations

| | Tissue | | Protein | | | | Protein | | |
|---|---|---|---|---|---|---|---|---|---|
| Cell line | type | Gene | change | Classification | Type | Gene 2 | change 3 | Classification 4 | Type5 |
| 22RV1 | Prostate | BRCA2 | p.V1810I | Missense_Mutation | SNP | BRCA2 | p.TK3030fs | Frame_Shift_Ins | INS |
| BT-474 | Breast | BRCA2 | p.S3094* | Nonsense_Mutation | SNP | BRCA1 | p.S2284L | Missense_Mutation | SNP |
| DU-145 | Prostate | BRCA1 | p.E962K | Missense_Mutation | SNP | | | | |
| HCC1954 | Breast | BRCA1 | p.V1810fs | Frame_Shift_Del | DEL | | | | |
| HCC38 | Breast | BRCA2 | p.K644R | Missense_Mutation | SNP | | | | |
| KURAMOCHI | Ovary | BRCA2 | p.R2318* | Nonsense_Mutation | SNP | | | | |
| MDA-MB-436 | Breast | BRCA1 | | Splice_Site | SNP | | | | |
| OC-316 | Ovary | BRCA2 | p.Q1782fs | Frame_Shift_Ins | INS | | | | |
| SNU-324 | Pancreas | BRCA2 | p.R2494* | Nonsense_Mutation | SNP | | | | |
| ZR-75-30 | Breast | BRCA2 | p.M2322I | Missense_Mutation | SNP | | | | |
| COV362 | Ovary | BRCA1 | | Splice_Site | SNP | | | | |
| JHOS-2 | Ovary | BRCA1 | p.M1V | Start_Codon_SNP | SNP | | | | |
| MDA-MB-468 | Breast | BRCA2 | p.M965I | Missense_Mutation | SNP | | | | |

Briefly, cells were thawed from a liquid nitrogen preserved state. Once cells have been expanded and divided at their expected doubling times, screening begins. Cells were seeded in growth media in black 384-well tissue culture treated plates and equilibrated via centrifugation. Post seeding, plates were placed in standard 5% CO₂ incubation pre- and post-compound treatment. At the time of treatment, a “time zero” set of assay plates (which do not receive treatment) were collected and ATP levels were measured using CellTiter-Glo 2.0 (Promega). Treatment combinations were collected using a 9×9 full dose matrix. The 9×9 full dose matrix includes: 8 dose points plus the no treatment control for the enhancee; 8 dose points plus the no treatment control for the enhancer; 64 combination ratio points. Compounds are added at time zero and will not be re-dosed. Two-fold and three-fold serial dilutions were carried out for onvansertib and olaparib, respectively, to reach a concentration of 0.7 nM for onvansertib (from 1500 nM to 0.7 nM) and 13.7 nM for olaparib (from 30 μM to 13.7 nM).

Treated assay plates were incubated with compound for 6 days. After the treatment time, plates were developed for endpoint analysis using CellTiter-Glo 2.0. Replicates of up to three were collected to account for assay variability. Data (inhibition and growth inhibition) were analyzed using software from Horizon Discovery.

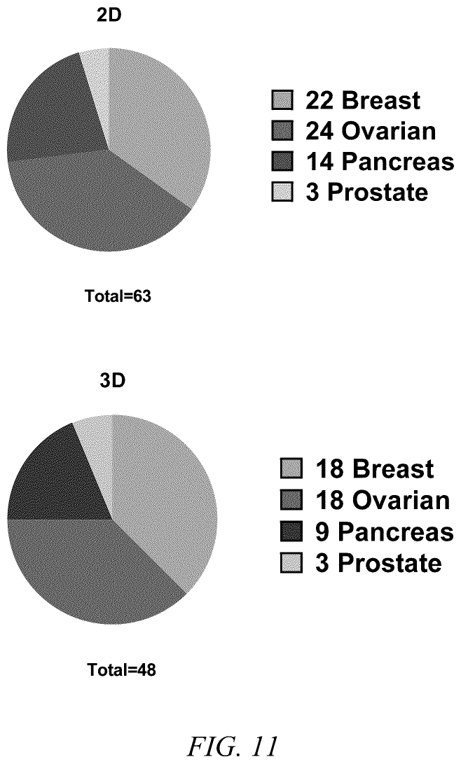

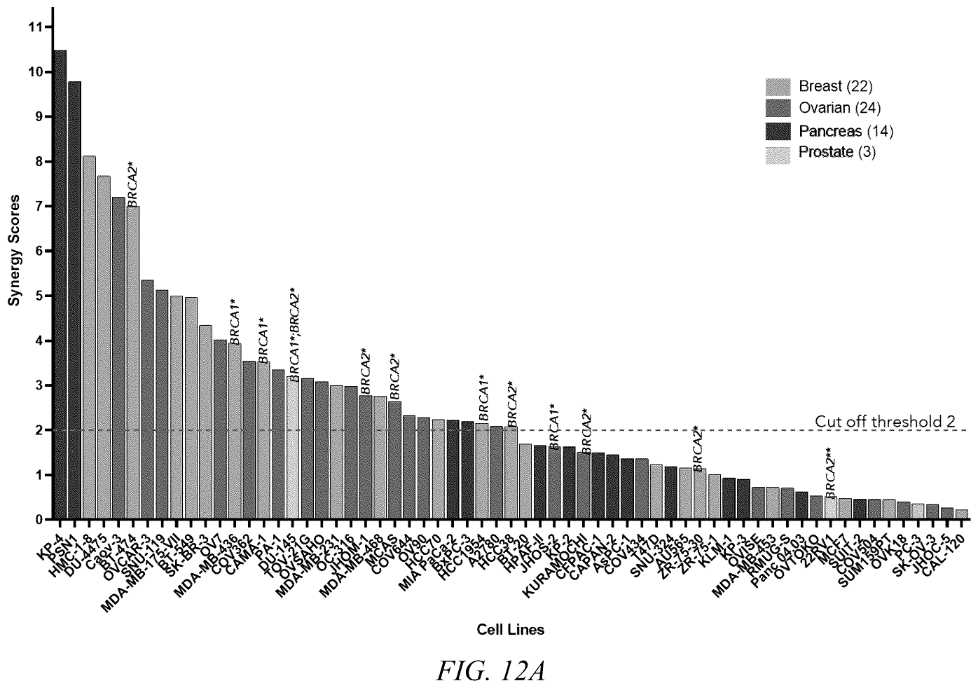

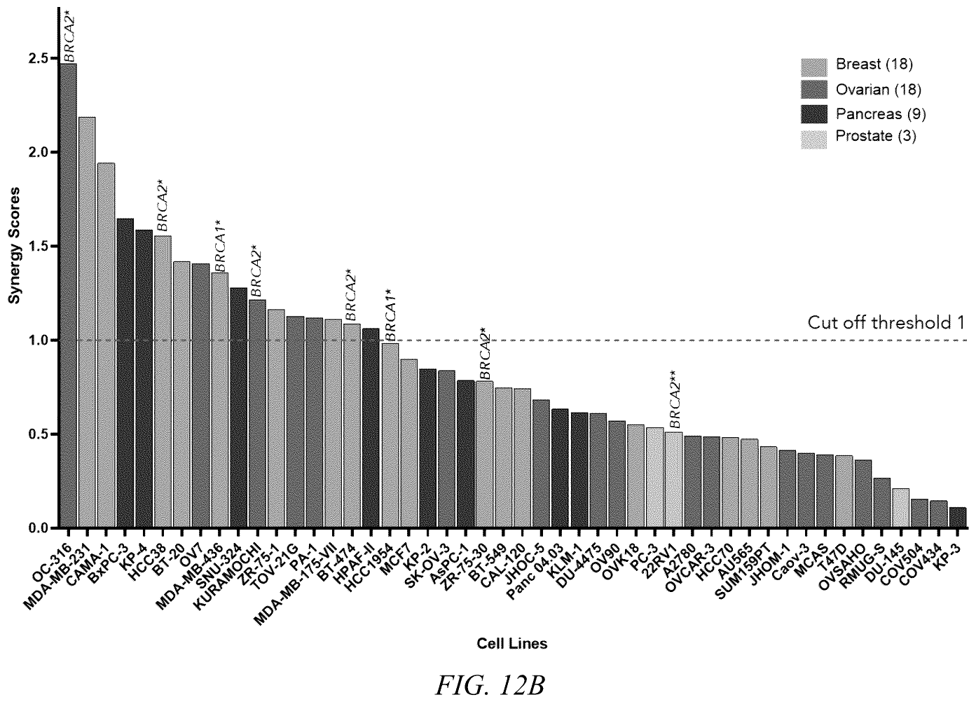

FIG. 11 depicts two circle graphs showing the cancer types of the cell lines tested in this example. 63 cell lines (22 breast cancer cell lines, 24 ovarian cancer cell lines, 14 pancreatic cancer cell lines and 3 prostate cancer cell lines) were tested in a two-dimensional cell culture (2D) and 46 cell lines (18 breast cancer cell lines, 18 ovarian cancer cell lines, 9 pancreatic cancer cell lines and 3 prostate cancer cell lines) in a three-dimensional cell culture (3D). The results of onvansertib and olaparib cell line screen in 2D and 3D is shown in FIG. 12A and FIG. 12B, respectively.

FIGS. 12A-B depict the synergy scores for each of the tested cell lines. As shown in the 2D cell line screen (FIG. 12A), 32 of the 63 cell lines tested (51%) had a synergy score greater than 2 and were considered potentially synergistic based on the manufactured guidance. The cell lines KP-4 (a human pancreatic ductal carcinoma cell line), PSN1 (human pancreatic adenocarcinoma cell line), HMC-1-8 (a target cell of the T-cell killer clone T (cHMC-1) derived from breast cancer), DU-4475 (breast ductal carcinoma cell line), Caov-3 (a primary ovarian cancer cell line with epithelial morphology), BT-474 (a cell line isolated from a solid, invasive ductal carcinoma from a breast cancer patient), OVCAR-3 (a high-grade serous ovarian adenocarcinoma cell line), and SNU-119 (a high grade serous ovarian cancer cell line) had a synergy score greater than 5. Synergy was observed in breast, ovarian, prostate and pancreatic cancer cell lines, and in both BRCA½ wild-type and mutant cell lines. 8 of the 12 cell lines (67%) with BRCA½ mutations had a synergy score higher than 2, including BT-474, MDA-MD-436 (a metastatic breast cancer cell line), COV362 (a high grade ovarian serous adenocarcinoma cell line), DU-145 (a prostate cancer cell line with epithelial morphology), OC-316 (an ovarian serous adenocarcinoma cell line), MDA-MD-468 (a cell line with epithelial morphology isolated from a patient with metastatic adenocarcinoma of the breast), HCC-1954 (an epithelial breast cancer cell line isolated from a primary stage IIA, grade 3 invasive ductal carcinoma with no lymph node metastases), and HCC38 (epithelial cells isolated from the mammary gland of a patient with a prior history of leiomyosarcoma). Information about each cell line used in the examples including origin, morphology, sequence variations, and disease type can be found, for example, at www.atcc.org and www.expasy.org.

As shown in the 3D cell line screen (FIG. 12B), 17 of the 48 cell lines tested (35%) had a synergy score greater than 1 (e.g., OC-316, MDA-MD-231, CAMA-1, BxPC-3, KP-4, HCC38, BT-20, OV7, MDA-MD-436, SNU-324, KURAMOCHI, ZR-75-1, TOV-21G, PA-1, MDA-MD-175-VII, BT-474, and HPAF-II) and were considered potentially synergistic based on the manufactured guidance. Synergy was observed in breast, ovarian and pancreatic cancer cell lines, and in both BRCA½ wild-type and mutant cell lines.

These results demonstrate that PLK1 inhibition can sensitize the activity of olaparib, leading to synergistic effect between onvansertib and olaparib.

### Example 7

### In Vitro Evaluation of Antitumor Activity of the Combination of Olaparib and Onvansertib

In this example, the activity of the combination of olaparib and onvansertib is evaluated in two different murine syngeneic ovarian cell lines: the ID8 system (Walton et al. 2016; Walton et al., 2017) and the recently obtained murine syngeneic model (Lyer et al.).

The ID8 system consists of ID8 murine cell line (p53 and BRCA½ wt), ID8 p53−/−(p53 deleted), ID8 p53−/−, BRCA1−/−(p53 and BRCA1 deleted) and ID8 p53−/−, BRCA2−/−(p53 and BRCA2 deleted). The murine syngeneic model system consists of BPPNM cells (TP53^{-/-R172H}, BRCA1^{-/}−; PTEN^{-/-}, NF1^{-/}−, Myc^{OE}); PPNM (TP53^{-/-R172H}, PTEN^{-/-}, NF1^{-/}−, Myc^{OE}) and BPCA (CCNE^{OE}, AKT2^{OE}, KRAS^{GD2V}.

Cytotoxic experiments are performed using MTS assays. Each cell line are treated with serial concentrations of the drugs (from 5 to 7 different drug concentrations) given concomitantly.

Treatments can last 72 hrs and cell survival is analyzed by the MTS assay system (Promega). MTS reagent is added on the cells and after a constant incubation time for all the plates absorbance is acquired using a plate reader (Infinite M200, TECAN). Data can be examined by isobologram analysis with Calcusyn Software (Biosoft, Cambridge, UK) and Combination Index (CI) values at the IC₅₀ can be calculated to assess the efficacy of the combination. All the experiments are done at least twice and with three replicates in each experimental group.

Based on the results, further studies can be conducted in the cell line where the combination demonstrates synergism. In particular, cell cycle analysis, in which cells not treated, treated with onvansertib, with olaparib, and with the combinations can be performed at different time points after the beginning of treatment (e.g., 8 hrs, 24 hrs, 48 hrs and 72 hrs). Briefly, about 2×10⁶ cells at the different time points can be fixed in ethanol 70%, stained and biparametric analysis can be done as previously described (Lupi et al.). For each sample, 10,000 events can be acquired with a FACS Calibur (Becton Dickinson, San Jose, CA) flow cytometer.

In addition, at these time points apoptosis can be evaluated (e.g., by caspase activation in cell extracts) and the status of DNA damage pathways (ATR/CHK1 axis), pRPA and pRAD51 can be investigated by western blot analysis along with activation of γH2AX and p-S10 histone H3.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating cancer, the method comprising: administrating a Poly-(ADP-ribose) polymerase (PARP) inhibitor and a Polo-like kinase 1 (PLK1) inhibitor to a subject with cancer, thereby inhibiting progression of the cancer.

2. The method of claim 1, wherein the cancer is ovarian cancer, breast cancer, prostate cancer, colorectal cancer, pancreatic cancer, or a combination thereof.

3. The method of any one of claims 1-2, wherein the cancer is a homologous recombination-deficient cancer.

4. The method of any one of claims 1-3, wherein the cancer is a BRCA1-mutant cancer, a BRCA2-mutant cancer, or both.

5. The method of any one of claims 1-4, wherein the PLK1 inhibitor and the PARP inhibitor are co-administered simultaneously.

6. The method of any one of claims 1-4, wherein the PLK1 inhibitor and the PARP inhibitor are administered sequentially.

7. The method of claim 6, wherein the PLK1 inhibitor is administered prior to the administration of the PARP inhibitor, and optionally wherein the PLK1 inhibitor is administered prior to the administration of the PARP inhibitor every day on which the subject is administered with the PLK1 inhibitor and the PARP inhibitor.

8. The method of claim 7, wherein the PLK1 inhibitor is administered about 30 minutes to about 5 hours prior to the administration of the PARP inhibitor on a given day.

9. The method of any one of claims 1-8, wherein the administration of the PLK1 inhibitor is oral administration, the administration of the PARP inhibitor is oral administration, or both.

10. The method of any one of claims 1-9, wherein the inhibition of cancer progression is greater than the combined inhibition of progression caused by the PARP inhibitor alone plus the PLK1 inhibitor alone.

11. The method of any one of claims 1-10, wherein the subject achieves a complete response.

12. The method of any one of claims 1-11, wherein the subject has received a prior PARP inhibitor treatment.

13. The method of any one of claims 1-12, wherein the subject did not respond to treatment with the PARP inhibitor alone.

14. The method of any one of claims 1-13, wherein the subject is known to be resistant to a PARP inhibitor therapy.

15. The method of any one of claims 1-14, wherein the PARP inhibitor and the PLK1 inhibitor are each administered to the subject in a cycle of at least twice or at least five times within a week.

16. The method of any one of claims 1-15, wherein the PARP inhibitor, the PLK1 inhibitor, or both are administered in a cycle of at least 7 days; optionally each cycle of treatment is at least about 21 days; and further optionally each cycle of treatment is from about 21 days to about 28 days.

17. The method of any one of claims 15-16, wherein the PLK1 inhibitor is administered on at least four days in the cycle.

18. The method of any one of claims 16-17, wherein the PLK1 inhibitor is not administered on at least one day in the cycle.

19. The method of any one of claims 1-18, wherein the PARP inhibitor is administered daily.

20. The method of claims 1-19, wherein the subject undergoes at least two cycles of the administration of the PARP inhibitor and the PLK1 inhibitor.

21. The method of any one of claims 1-20, wherein the PARP inhibitor is selective and/or specific for PARP inhibition.

22. The method of any one of claims 1-20, wherein the PARP inhibitor is iniparib (BSI 201), talazoparib (BMN-673), olaparib (AZD-2281), AZD5305, NMS-293, rucaparib (AG014699, PF-01367338), ABT-888, Veliparib (ABT-888), niraparib, CEP 9722, MK 4827, BGB-290 (pamiparib), BSI-201, CEP-8983, E7016, 3-aminobenzamide, or a combination thereof; optionally the PARP inhibitor is olaparib.

23. The method of any one of claims 1-22, wherein the PLK1 inhibitor is selective and/or specific for PLK1.

24. The method of any one of claims 1-22, wherein the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof.

25. The method of any one of claims 1-22, wherein the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280; and optionally the PLK1 inhibitor is onvansertib.

26. The method of claim 25, wherein onvansertib is administered at 12 mg/m²-90 mg/m².

27. The method of any one of claims 25-26, wherein a maximum concentration (Cₘₐₓ) of onvansertib in a blood of the subject is from about 100 nmol/L to about 1500 nmol/L.

28. The method of any one of claims 25-27, wherein an area under curve (AUC) of a plot of a concentration of onvanserib in a blood of the subject over time is from about 1000 nmol/L.hour to about 400000 nmol/L.hour.

29. The method of any one of claims 25-28, wherein a time (Tₘₐₓ) to reach a maximum concentration of onvansertib in a blood of the subject is from about 1 hour to about 5 hours.

30. The method of any one of claims 25-29, wherein an elimination half-life (T_{1/2}) of onvansertib in a blood of the subject is from about 10 hours to about 60 hours.

31. The method of any one of claims 1-30, wherein the PARP inhibitor is olaparib and the PLK1 inhibitor is onvansertib.

32. The method of any one of claims 1-31, wherein the subject has received at least one prior cancer treatment, and optionally wherein the prior treatment does not comprise the use of a PARP inhibitor, a PLK inhibitor, or both.

33. The method of any one of claims 1-32, wherein the subject was in remission for cancer, optionally wherein the subject in remission for cancer was in complete remission (CR) or in partial remission (PR).

34. The method of any one of claims 1-33, further comprising one or more of (1) determining cancer status of the subject, (2) determining responsiveness of the subject to a PLK1 inhibitor treatment, and (3) administering one or more cancer therapeutics or therapies for the cancer.

35. The method of any one of claims 1-34, the subject is human.

36. A method of sensitizing cancer cells to a PARP inhibitor, the method comprising: contacting cancer cells with a composition comprising a Polo-like kinase 1 (PLK1) inhibitor, thereby sensitizing the cancer cells to the PARP inhibitor.

37. The method of claim 36, wherein the PLK1 inhibitor is onvansertib, the PARP inhibitor is olaparib, or both.

38. The method of any one of claims 36-37, wherein contacting cancer cells with the composition occurs in vitro, ex vivo, and/or in vivo.

39. The method of any one of claims 36-38, wherein contacting cancer cells with the composition is in a subject, and optionally wherein the subject did not respond to, or is known to be resistant to, the PARP inhibitor.

40. The method of claim 39, wherein the subject had prior treatment with the PARP inhibitor.

41. The method of any one of claims 39-40, wherein the subject is a mammal, and optionally the mammal is human.

42. The method of any one of claims 36-41, comprising determining sensitization of the cancer cells to the PARP inhibitor after being contacted with the composition.

43. The method of any one of claims 36-42, comprising contacting the cancer cells with the PARP inhibitor, and optionally wherein contacting the cancer cells with the PARP inhibitor occurs in the subject.

44. The method of claim 43, comprising determining the response of the subject to the PARP inhibitor.

45. The method of any one of claims 43-44, wherein contacting the cancer cells with the PARP inhibitor is concurrent with the contacting the cancer cells with the composition, or after the contacting the cancer cells with the composition.

46. A kit, comprising
a Polo-like kinase 1 (PLK1) inhibitor; and
a manual providing instructions for co-administrating the PLK1 inhibitor with a Poly-(ADP-ribose) polymerase (PARP) inhibitor to a subject for treating cancer.

47. The kit of claim 46, wherein the subject has ovarian cancer, breast cancer, prostate cancer, colorectal cancer, pancreatic cancer, or a combination thereof.

48. The kit of claim 46 or 47, wherein the cancer is a homologous recombination (HR)-deficient cancer.

49. The kit of any one of claims 46-48, wherein the cancer is a BRCA1-mutant cancer, a BRCA2-mutant cancer, or both.

50. The kit of any one of claims 46-49, wherein the instructions comprise instructions for co-administrating the PLK1 inhibitor and the PARP inhibitor simultaneously.

51. The kit of any one of claims 46-49, wherein the instructions comprise instructions for co-administrating the PLK1 inhibitor and the PARP inhibitor sequentially.

52. The kit of any one of claims 46-51, wherein the instructions comprise (1) instructions for administering of the PLK1 inhibitor orally, (2) instructions for administrating the PARP inhibitor orally, or both.

53. The kit of any one of claims 46-52, wherein the instructions comprise instructions the subject has received a prior PARP inhibitor treatment.

54. The kit of any one of claims 46-53, wherein the instructions comprise instructions the subject did not respond to treatment with the PARP inhibitor alone.

55. The kit of any one of claims 46-54, wherein the instructions comprise instructions the subject is known to be resistant to a PARP inhibitor therapy.

56. The kit of any one of claims 46-55, wherein the instructions comprise instructions for administering each of the PARP inhibitor and the PLK1 inhibitor to the subject in a cycle of at least twice or at least five times within a week.

57. The kit of any one of claims 46-56, wherein the instructions comprise instructions for administering the PARP inhibitor, the PLK1 inhibitor, or both are in a cycle of at least 7 days; and optionally wherein each cycle of treatment is at least about 21 days, and further optionally each cycle of treatment is from about 21 days to about 28 days.

58. The kit of claim 57, wherein the instructions comprise instructions for administering the PLK1 inhibitor on at least four days in the cycle.

59. The kit of any one of claims 57-58, wherein the instructions comprise instructions for not administering the PLK1 inhibitor on at least one day in the cycle.

60. The kit of any one of claims 36-59, wherein the instructions comprise instructions for administrating the PARP inhibitor daily.

61. The kit of any one of claims 46-60, wherein the instructions comprise instructions for administrating the PARP inhibitor and the PLK1 inhibitor for at least two cycles.

62. The kit of any one of claims 46-61, wherein the PARP inhibitor is selective and/or specific for PARP1 and/or PARP2 inhibition.

63. The kit of any one of claims 46-62, wherein the PARP inhibitor is iniparib (BSI 201), talazoparib (BMN-673), olaparib (AZD-2281), AZD5305, rucaparib (AG014699, PF-01367338), ABT-888, veliparib (ABT-888), niraparib, CEP 9722, MK 4827, BGB-290 (pamiparib), BSI-201, CEP-8983, E7016, 3-aminobenzamide, NMS-P293, or a combination thereof; and optionally wherein the PARP inhibitor is olaparib.

64. The kit of any one of claims 46-63, wherein the PLK1 inhibitor is selective and/or specific for PLK1.

65. The kit of any one of claims 46-64, wherein the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof.

66. The kit of any one of claims 46-64, wherein the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280; and optionally wherein the PLK1 inhibitor is onvansertib.

67. The kit of claim 66, wherein the instructions comprise instructions for administering onvansertib at 12 mg/m²-90 mg/m².

68. The kit of any one of claims 46-67, wherein the PARP inhibitor is olaparib and the PLK1 inhibitor is onvansertib.

69. The kit of any one of claims 46-68, wherein the instructions comprise instructions the subject has received at least one prior treatment for the cancer, and optionally wherein the prior treatment does not comprise the use of a PARP inhibitor, a PLK inhibitor, or both.

70. The kit of any one of claims 46-69, wherein the instructions comprise instructions the subject was in remission for cancer, and optionally wherein the subject in remission for cancer was in complete remission (CR) or in partial remission (PR).

71. The kit of any one of claims 46-70, further comprising the PARP inhibitor.
